(19) United States
(12) Patent Application Publication
(10) Pub. No.: US 2026/0201295 A1
(43) Pub. Date: Jul. 16, 2026

(54) Customizable Microfluidic Platform with Perfusable Microvasculature and 3D Lattice Scaffolds for In-Vitro Brain Models

(71) Applicant: Board of Trustees of the University of Arkansas, Little Rock, AR (US)

(72) Inventors: Nathaniel Harris, Springdale, AR (US); Min Zou, Fayetteville, AR (US)

(21) Appl. No.: 19/451,397

(22) Filed: Jan. 16, 2026

Related U.S. Application Data
(60) Provisional application No. 63/746,073, filed on Jan. 16, 2025.

(51) Int. Cl. C12M 3/06; C12M 1/00; C12M 1/12; C12M 1/32; C12M 3/00; G06F 30/10; G06F 30/23; G06T 17/20

(52) U.S. Cl. CPC C12M 23/16; C12M 23/12; C12M 23/38; C12M 23/48; C12M 25/02; C12M 25/14; C12M 29/10; C12M 29/14; C12M 29/18; G06F 30/10; G06F 30/23; G06T 17/20; G06T 2210/41

(57) ABSTRACT

A microfluidic chip for use in in-vitro brain models is disclosed. The chip includes a microcapillary scaffold having a scaffold base and a scaffold vasculature. The chip includes a plate having wells, and the microcapillary scaffold is secured in the well. The chip also includes a microfluidic interface system attached to the microcapillary scaffold, which includes heat-shrink tubing and hypodermic needles. An external fluidic system is connected to the microfluidic interface system for supplying fluid for perfusion through the microcapillary scaffold. The microcapillary scaffold includes a customizable capillary membrane and a customizable polygonal support or a lattice support. The scaffold base is made from a rigid hybrid polymer resin, while the scaffold vasculature is made from a rigid hybrid polymer resin, a PEGDA resin or an elastomeric resin.

### CROSS-REFERENCE TO RELATED APPLICATIONS

This application claims the benefit of U.S. Provisional Application No. 63/746,073, entitled “Customizable Vascularized Lattice for In-vitro Brain Models,” filed on Jan. 16, 2025. The complete disclosure of said provisional application is hereby incorporated by reference.

### STATEMENT REGARDING FEDERALLY SPONSORED RESEARCH OR DEVELOPMENT

Not applicable

### BACKGROUND OF THE INVENTION

Three-dimensional (3D) in vitro models are increasingly essential for investigating human brain development, disease mechanisms, and drug responses. Compared to traditional two-dimensional (2D) cultures, which fail to replicate spatial organization and physiological interactions, 3D models better mimic the complex microenvironments of native tissues. Advances in organoids, hydrogel scaffolds, and 3D bioprinting have expanded neural tissue modeling capabilities. However, a critical limitation persists: the lack of perfusable microvasculatures.

The brain's microvascular network plays an indispensable role in maintaining tissue viability and homeostasis. In the absence of vascularization, brain organoids are restricted in size and often develop necrotic cores due to limited oxygen and nutrient diffusion. Human brain capillaries are exceptionally narrow (7-10 μm in diameter) and densely spaced (~25 μm apart), forming tortuous pathways essential for efficient exchange and blood-brain barrier (BBB) support. The BBB restricts passage of nearly all large molecules and up to 98% of small molecules, presenting additional challenges for central nervous system drug delivery. Moreover, the lack of physiological flow and endothelial interaction in existing models limits their utility in replicating neurovascular dynamics and investigating disease pathogenesis.

To address these limitations, various strategies have been explored to introduce microvasculature into 3D models. Self-organized endothelial networks offer biological integration but often suffer from poor reproducibility and lack physiological organization. Traditional microfluidic platforms provide precise flow control, yet their planar, oversized channels fail to emulate the complex and capillary-scale architecture of native brain microvasculature.

Two-photon lithography (TPL) offers sub-micron resolution and user-defined 3D geometries, making it a promising approach for constructing capillary-scale scaffolds. Prior studies have used TPL to fabricate microvascular and BBB models. However, these models primarily employ rigid photoresists and simple straight tube designs, resulting in quasi-2D structures with limited spatial complexity. They are unable to replicate the tortuous, branching, and arbitrarily oriented capillary geometries required to truly mimic brain microvasculature in three dimensions. Furthermore, they lack surrounding 3D lattice structures that enable neural cells to distribute and organize naturally in three dimensions around perfusable capillaries—a requirement for neurovascular unit (NVU) formation and functional neurovascular modeling. Practical issues with perfusion interfaces, often relying on bulky, non-integrated connectors, also hinder their adoption for long-term and biologically integrated studies.

In this invention, we introduce a versatile and modular platform that addresses these challenges through three key innovations. First, a mesh-based design workflow in Rhinoceros 3D enables generation of arbitrarily routed 3D capillary scaffolds with tunable pore geometries and integrated mesoscale supports. These lattices not only stabilize soft capillary structures but also provide 3D environments to support neural cell attachment, distribution, and organization. Second, multi-material TPL fabrication using rigid (OrmoComp), intermediate stiffness (polyethylene glycol diacrylate, PEGDA 700), and soft elastomeric (IP-polydimethylsiloxane (PDMS)) materials enables tuning of scaffold mechanical properties across a wide range, balancing geometric fidelity and biological compliance. Rigid OrmoComp ensures precision and stability, PEGDA 700 offers a synthetic hydrogel to mimic a natural extracellular matrix environment, and IP-PDMS enables architected structures with unmatched softness. Third, a simple microfluidic interface based on heat-shrink tubing and hypodermic needles eliminates bulky fittings and facilitates leak-free perfusion. Together, these advances establish a customizable platform capable of supporting perfusable, biomimetic microvasculature systems for neural cell culture, neurovascular co-culture, and advanced brain-on-chip models. This system holds broad potential for applications in neurodegenerative disease modeling, BBB permeability studies, and vascularized organoid development.

### BRIEF SUMMARY OF THE INVENTION

Perfusable microvasculature is critical for advancing in vitro tissue models, particularly for neural applications where limited diffusion impairs organoid growth and fails to replicate neurovascular function. This study presents a versatile fabrication platform that integrates mesh-driven design, TPL, and modular interfacing to create multi-material, perfusable 3D microvasculatures. Various 2D and 3D capillary paths were test-printed using both polygonal and lattice support strategies. A double-layered capillary scaffold based on the Hilbert curve was used for comparative materials testing. Methods for printing rigid (OrmoComp), moderately stiff hydrogel (polyethylene glycol diacrylate, PEGDA 700), and soft elastomeric (photocurable polydimethylsiloxane, PDMS) were developed and evaluated. Cone support structures enabled high-fidelity printing of the softer materials. A compact heat-shrink tubing interface provided leak-free perfusion without bulky fittings. Physiologically relevant flow velocities and Dextran diffusion through the scaffold were successfully demonstrated. Cytocompatibility assays confirmed that all TPL-printed scaffold materials supported human neural stem cell viability. Among peripheral components, lids fabricated via fused deposition modeling designed to hold microfluidic needle adapters exhibited good biocompatibility, while those made using liquid crystal display-based photopolymerization showed significant cytotoxicity despite indirect exposure. Overall, this platform enables the design and fabrication of tunable microvascular systems for neurovascular modeling, blood-brain barrier studies, and integration into vascularized organ-on-chip applications.

In one preferred embodiment, the present invention is directed to a microfluidic chip for use in in-vitro brain models. The microfluidic chip includes a microcapillary scaffold having a scaffold base and a scaffold vasculature. The chip also includes a plate having wells, and the microcapillary scaffold is secured in one of the wells. A microfluidic interface system is attached to the microcapillary scaffold, which includes heat-shrink tubing and hypodermic needles. An external fluidic system is connected to the microfluidic interface system for supplying fluid for perfusion through the microcapillary scaffold. The microcapillary scaffold includes a capillary membrane and a polygonal support or a lattice support. The scaffold base is made from a rigid hybrid polymer resin, while the scaffold vasculature may be made from a rigid hybrid polymer resin, a PEGDA resin, an elastomeric resin, or other extracellular matrix (ECM)-mimetic materials.

In another preferred embodiment, the present invention is directed to a method of making a microfluidic chip for use in in-vitro brain models. The method includes the steps of: (1) fabricating a microcapillary scaffold using two photon lithography, wherein the microcapillary scaffold comprises a scaffold base and a scaffold vasculature, wherein the scaffold base comprises a first port and a second port; (2) attaching a microfluidic interface system to the microcapillary scaffold, wherein the microfluidic interface system includes a first heat-shrink tubing, a first hypodermic needle, a second heat-shrinking tubing and a second hypodermic needle, wherein the step of attaching the microfluidic interface system includes the steps of attaching a first end of the first heat-shrink tubing to the first port of the scaffold base and applying heat sufficient to seal the first end of the first heat-shrink tubing to the first port, attaching a first end of the second heat-shrink tubing to the second port of the scaffold base and applying heat sufficient to seal the second end of the second heat-shrink tubing to the second port, inserting the first hypodermic needle in a second end of the first heat-shrink tubing, and inserting the second hypodermic needle in a second end of the second heat-shrink tubing; and (3) securing the microcapillary scaffold in a well of a plate. The method also includes the step of creating a design of the microcapillary scaffold before fabricating the microcapillary scaffold using three-dimensional modeling software. The microcapillary scaffold includes a capillary membrane and a polygonal support or a lattice support. A rigid hybrid polymer resin is used to fabricate at least some of the microcapillary scaffold. PEGDA resin or an elastomeric resin may also be used to fabricate some of said microcapillary scaffold. Alternatively, ECM-mimetic materials may be used.

These and other features, objects and advantages of the present invention will become better understood from a consideration of the following detailed description of the preferred embodiments and appended claims in conjunction with the drawings as described following.

The following abbreviations are used in this application: TPL: Two photon lithography; PGMEA: Propylene Glycol Monomethyl Ether Acetate; IPA: Isopropyl Alcohol; PLA: Polylactic acid; LCD: Liquid crystal display; BBB: Blood-brain barrier; NVU: Neurovascular unit; PEGDA: Polyethylene glycol diacrylate; NURBS: Non-uniform rational b-spline; FOV: Field of view; CAD: Computer aided design; DiLL: Dip-in laser lithography; bFGF: Basic fibroblast growth factor; EGF: Endothelial growth factor; UV: Ultraviolet; PBS: Phosphate buffered saline; SEM: Scanning electron microscope; DIV: Days in vitro.

### BRIEF DESCRIPTION OF THE DRAWINGS

FIGS. 1A-11 illustrate design workflow and support strategies for versatile microcapillary scaffolds design platform. FIG. 1A shows capillary membrane pore generation, including equations used for calculating the surface subdivisions to achieve the desired number of subdivisions and their side lengths. FIG. 1B shows polygonal support structure design. Two-dimensional examples of the polygonal support strategy are shown, including a 2D Hilbert curve with supports at FOV intersections shown in FIG. 1C and a fourth-degree planar Hilbert microcapillary scaffold shown in FIG. 1D. Three dimensional designs using the polygonal support strategy include a 3D second-degree Hilbert curve shown in FIG. 1E and double helix designs with equidistant supports shown in FIG. 1F. FIG. 1G is a schematic of lattice-based support method for 3D capillary designs based on a truncated octahedron unit cell. Design examples using the lattice-based support method include a 3D second-degree Hilbert scaffold shown in FIG. 1H and double helix designs shown in FIG. 1I.

FIGS. 2A-2G are schematics showing the 3D printing of microcapillary scaffolds using TPL. Single-step TPL printing process using OrmoComp for both the base and the vasculature components is shown in FIG. 2A. Uncrosslinked OrmoComp was removed using baths of PGMEA shown in FIG. 2B and IPA shown in FIG. 2C. First stage of dual-step TPL printing process using OrmoComp for the base components is shown in FIG. 2D. In-place development of the OrmoComp using PGMEA is shown in FIG. 2E. Stage two of dual-step TPL printing process using PEGDA or IP-PDMS to print the vasculature components is shown in FIG. 2F. The inset shown is a top-down view of the corner fiducial mark printed for alignment after resin application. PEGDA or IP-PDMS shown in FIG. 2F were developed in IPA after printing as shown in FIG. 2G.

FIGS. 3A-3F are schematics of the microfluidic interface and pumping setup facilitated via heat-shrink tubing interface. FIG. 3A is a schematic of heat shrink tubing aligned to the TPL-printed nozzles without heat, FIG. 3B is a schematic of application of heat to shrink the tubing around the nozzles, and FIG. 3C is a schematic of UV crosslinking OrmoComp to seal the connection. FIG. 3D is a top-down view and FIG. 3E is a side view of design of the 3D printed hypodermic needle holder. The 3D printed needle holder is UV crosslinked with OrmoComp onto the top of the well of a 12 well plate. The TPL-printed scaffold is secured to the bottom of the well following forming the heat shrink tubing connections. The hypodermic needles are then inserted into the heat shrink tubing ends through the capillary needle inlet and secured in place via heat and OrmoComp resin. This creates a sealed interface for perfusion of the capillary membrane. A second set of needles is used to exchange the medium on the outside of the membrane. A removable glass lid is used to keep the chamber from evaporating. Schematic of the pump setup is shown is FIG. 3F, including capillary perfusion via syringe pump and external exchange via peristaltic pump. Capillary perfusion is accomplished by modifying a linear syringe pump to perform push and pull actions in the same motion. The 12 well plate is observed in situ using a small format microscope.

FIGS. 4A-4L show design optimization for two-dimensional microvasculatures printed in OrmoComp. FIG. 4A is a 3D model of a second-degree Hilbert curve vasculature design with 21 support rings evenly spaced along the path length of the curve. FIG. 4B is a close-up view of the pores. FIG. 4C is an isometric SEM image showing the test print. SEM micrographs showing misaligned sections due to unsupported geometry at the FOV intersections and closed pores on the sides is shown in FIG. 4D, and a top view of misaligned sections at FOV intersections is shown in FIG. 4E. FIG. 4F is a 3D model of the same Hilbert curve design using 25 support rings placed at FOV intersections. FIG. 4G is a close-up view of elongated pores on the sides of the vasculature model. SEM micrographs showing the isometric view of the test print is shown in FIG. 4H, open pores along the sides of the vasculature membrane is shown in FIG. 4I, and the top view of aligned geometry at FOV intersections due to appropriately placed supports is shown in FIG. 4J. Renders of pore diameter distribution along Hilbert curve is shown in FIG. 4K, with magnified view of the bottom right of FIG. 4K showing in FIG. 4L.

FIGS. 5A-5L show the design and TPL test prints of 2D and 3D microvascular scaffolds with integrated lattice support, all fabricated using OrmoComp. FIG. 5A shows the Rhino model, FIG. 5B shows the isometric SEM view, and FIG. 5C shows a close-up of pores of the 2H Hilbert curve. FIG. 5D shows the Rhino model, FIG. 5E shows the isometric SEM view, and FIG. 5F shows the pore details of the stacked 2D Hilbert curves. FIG. 5G shows the Rhino model, FIG. 5H shows an isometric SEM view, and FIG. 5I shows a pore close-up of the helical scaffold. FIG. 5J shows the Rhino model, FIG. 5K shows the isometric SEM view, and FIG. 5L shows the pore close-up of the 3D second-degree Hilbert curve.

FIGS. 6A-6C are SEM micrographs of test printed OrmoComp chips. FIG. 6A shows a flat base without mesoscale supports, FIG. 6B shows a base with cone supports, and FIG. 6C shows a close-up view of the cone supports. The capillary tube membrane geometry was subtracted from the cones to ensure the chips could still be perfused.

FIGS. 7A-7P are SEM images and registration analysis of microcapillary scaffolds fabricated from different materials with and without cone supports. FIGS. 7A, 7D, 7G, 7J, and 7M are SEM images of scaffolds made from OrmoComp, PEGDA 700, and IP-PDMS on flat or cone-supported bases. FIGS. 7B, 7E, 7H, 7K, and 7N are overlap images comparing SEM results to Rhino-generated binary masks. FIGS. 7C, 7F, 7I, 7L, and 7O are close-up views showing edge deformation on flat bases and improved geometry control with cone supports. FIG. 7P is a quantitative registration analysis showing overlap versus total print error.

FIGS. 8A-8F show the fabrication and testing setup for capillary chips. FIG. 8A is a close-up view of a nozzle with the tubing fractured away. FIG. 8B is an overall view of an OrmoComp chip. FIG. 8C is a close-up view of the nozzle with the tubing sealed in place with OrmoComp. FIG. 8D shows an assembly of heat shrink tubing onto OrmoComp, PEGDA 700, and IP-PDMS scaffolds. FIG. 8E shows an overview of a fabricated chip and hypodermic needle holder. FIG. 8F shows an in-situ testing setup, including a small format microscope equipped with a custom three-axis stage, a syringe pump equipped for both push and pull functions, and a peristaltic pump for external flow for media exchange.

FIGS. 9A-9B shows an assessment of OrmoComp, PEGDA 700, and IP-PDMS scaffolds under cell culture imaging conditions. FIG. 9A are brightfield images of dry and aqueous conditions. FIG. 9B are dry fluorescence imaging tests using blue, green, and red filter cube sets.

FIGS. 10A-10C show Dextran diffusion experiments in OrmoComp chips. FIG. 10A is a fluorescence image prior to Dextran diffusion. FIG. 10B shows diffusion of Dextran using the push-pull method. FIG. 10C shows extraction of Dextran through membrane in chip, showing interior of double-layered Hilbert curve chip.

FIGS. 11A-11E show particle flow speed measurements. FIG. 11A is a 3D rendering of a solid-wall straight microchannel with a 40 μm inner diameter and 1 mm length, shown in isometric and cutaway views. FIG. 11B is a schematic of the push-flow setup used to drive bead perfusion through the microchannel. FIG. 11C is a maximum intensity projection of particle image velocimetry (PIV) time-lapse frames overlaid on phase contrast background, illustrating complete particle paths. FIG. 11D is an example image of fluorescent particles in flow during a single frame. FIG. 11E shows a tracked particle paths plotted from the PIV analysis.

FIGS. 12A-12K show results of a viability assay of hNSCs at DIV 3 on a microfluidic chip and TPL materials. FIGS. 12A-I are example 10× images of the assay: TCP control (FIG. 12A), 3D printed needle holder materials including Anycubic Transparent Green LCD resin (FIG. 12B), Siraya Tech Blu resin (FIG. 12C), PLA polymer (FIG. 12D), heat shrink tubing (FIG. 12E), Ormoprime (FIG. 12F), OrmoComp (FIG. 12G), PEGDA (FIG. 12H), and IP-PDMS (FIG. 12I). FIG. 12J shows the average viability of hNSCs on each material. FIG. 12K shows the average live cell density of hNSCs on each material.

FIGS. 13A-13D are design examples of branched vascular structures. A separate process is used to convert these vascular designs into continuous porous membranes. Fluid can be pumped through multiple channels using a secondary printed holder and adapter (purple; periphery). Human brain microvascular endothelial cells (hBMECs) can be seeded on the inner walls of the vascular membrane tubes (red; center). The system accommodates various vascular designs, including regular repeating lattices (FIGS. 13A-13B) or bio-inspired Voronoi lattices (FIG. 13C-13D). The larger inlet and outlet branches mimic the function of arterioles and venules, feeding the in-vivo capillary beds.

FIGS. 14A-14D illustrate a coaxial membrane designed to enable selective pericyte seeding between astrocytes (parenchyma) and endothelial cells (vessel). FIG. 14A is a schematic of the neurovascular unit (NVU) cellular structure. FIG. 14B is a side view and FIG. 14C is an oblique angle view of the coaxial porous membrane (also shown in FIG. 15B) surrounding the microvasculature (also shown in FIG. 15A), providing a compartmentalized space for pericytes, essential for maintaining NVU integrity. The Crystallon add-in was used to generate support beams (also shown in FIG. 15A) that stabilize the capillary and coaxial membranes, ensuring proper alignment of cellular structures while maintaining open channels for fluid flow. These membranes are supported by lattice structures (also shown in FIG. 15B) that facilitate the differentiation of neural stem cells into neurons and astrocytes. FIG. 14D shows a biomimetic design printed via TPL, corresponding to the design in FIG. 14C.

FIGS. 15A-15B are detailed view of individual components from FIG. 14C. FIG. 15A illustrates the microvasculature and support beams. FIG. 15B illustrates the coaxial porous membrane and an aECM scaffold designed to support the differentiation and organization of neurons and astrocytes.

FIGS. 16A-16D illustrate human neural stem cell (hNSC) differentiation on two scaffold designs. FIG. 16A shows a 2D Hilbert curve with simple supporting structures. FIG. 16B shows a 2D Hilbert curve with lattice supporting structures. FIGS. 16C-16D are confocal microscopy images with immunostaining that reveal that neurons (stained with β-III Tubulin) predominantly develop on the scaffold's 3D surfaces, while astrocytes (stained with GFAP) primarily differentiate on the flatter regions. This design allows for customization of the scaffold's structure to achieve the desired ratio of astrocytes to neurons, tailored to specific brain regions being modeled.

FIGS. 17A-17B are schematics showing that the scaffold can be engineered to have micro- and nanoscale topographical cues to guide the organization, growth, and alignment of cells, ensuring the tissue model mimics in-vivo conditions. For example, FIG. 17C shows 3D scaffold in the center area promoted neural stem cell differentiate into neurons, while surrounding flat areas favored astrocyte differentiation. FIG. 17D shows nanoscale islands promoted neural stem cells differentiate into neurons, while the adjacent flat area favored astrocyte differentiation.

FIGS. 18A-18B are SEM analysis of human neural stem cells (hNSCs) differentiated on TPL-fabricated scaffolds at DIV 14 (FIG. 18A) and DIV 28 (FIG. 18B). FIGS. 18C-18D are confocal laser scanning microscopy images for DIV 14 (FIG. 18C) and DIV 28 (FIG. 18D) confirm the identity of cells on the support structure (*) and those extending projections between the capillary tube and the support structure (**) as astrocytes. Meanwhile, denser networks of cells located on the capillary tubes and around the mouth of the capillary tube (***) are identified as neurons.

FIGS. 19A-19D illustrate compartmentalized channels for fluid flow and chemical stimulation. FIGS. 19A-19B are top-down views and FIGS. 19C-19D are oblique angle views of the Dodecahedron and Voronoi lattice designs, respectively. The scaffold incorporates compartmentalized microfluidic channels that allow for precise control over the delivery of fluids, nutrients, cells, and chemical agents, such as alcohol, caffeine, or therapeutic drugs. These channels replicate the fluid dynamics of the brain's native microenvironment, ensuring continuous perfusion and maintaining cell viability. For instance, four distinct channels can seed different cell types at specific locations within the scaffold: neurons, astrocytes, microglia, and human neural stem cells. The flexible fabrication process allows channels to branch and deposit cells across multiple levels. These channels also facilitate localized media exchanges, growth factor deposition, and media sampling, enhancing the scaffold's functionality for more precise experimental analysis.

FIGS. 20A-20B are fluorescence flow tests using 1 μm diameter green fluorescent polystyrene microparticles in the center capillary membrane and near-infrared (NIR) dye (CF633) in the coaxial outer membrane. FIG. 20A is a fluorescence microscopy image overlaid with brightfield, green, and red channels during testing in air. FIG. 20B is a red-green (RG) intensity profile across the membrane, showing the fluorescence distribution. The intensity profiles demonstrate good alignment with the expected location of each fluorescent marker, validating independent flow within each membrane channel.

### DETAILED DESCRIPTION OF THE INVENTION

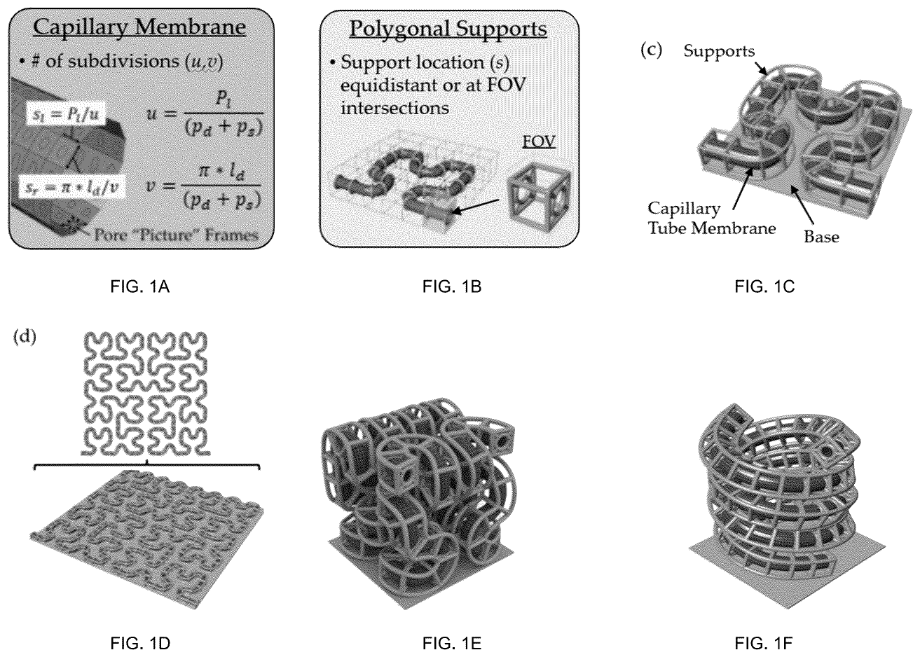

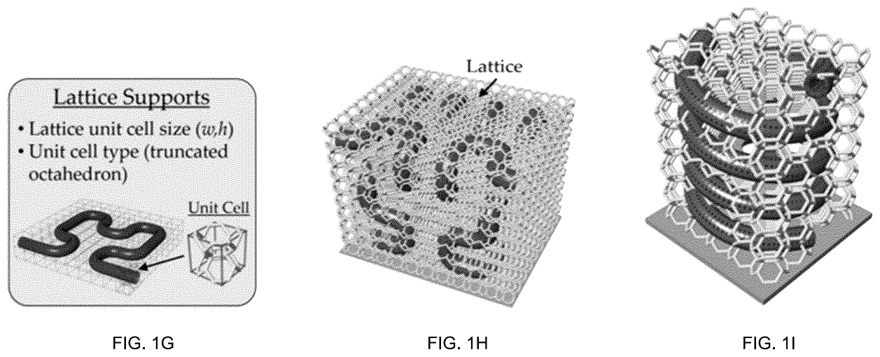

With reference to FIGS. 1A-20B, the preferred embodiments of the present invention may be described.

Materials and Methods: Path-Adaptable Microcapillary Design Platform: To generate complex and physiologically relevant microcapillary geometries, we developed a versatile scaffold design pipeline using Rhinoceros 3D (Rhino 7) along with the Grasshopper plugin. This platform enables rapid generation of microcapillary models along arbitrary paths, with tunable parameters including lumen diameter, pore diameter, pore spacing, wall thickness, and support beam thickness. Microcapillary paths are imported from comma-separated value (.csv) files containing 3D coordinates, which define a Non-Uniform Rational B-Spline (NURBS) curve used as the central axis of the capillary geometry. Pores in the surface of the microcapillary's cylindrical lumen are created using mesh-based operations rather than traditional model tree-based CAD modeling. A parametric domain subdivision of the cylindrical capillary surface sets the spacing and location of pores (FIG. 1A).

Two equations control the pore distribution along the capillary tube surface, including:

$$u = \frac{{P}_{l}}{( {p}_{d} + {p}_{s} )} \qquad ( 1 )$$

$$v = \frac{π * {l}_{d}}{( {p}_{d} + {p}_{s} )} \qquad ( 2 )$$

where u in Eq. (1) defines the number of subdivisions along the capillary tube path length (Pₗ) based on the desired pore diameter (p_{d}) and desired pore spacing (pₛ). Similarly, v in Eq. (2) defines the number of subdivisions in the circumferential direction for the desired lumen diameter (l_{d}), pore diameter, and pore spacing. Therefore, the side lengths of each surface subdivision are determined using the following:

$${s}_{l} = \frac{{P}_{l}}{u} \qquad ( 3 )$$

$${s}_{r} = \frac{π * {l}_{d}}{v} \qquad ( 4 )$$

where Eq. (3) defines the length along the capillary membrane path direction (sₗ), and Eq (4) defines the length along the capillary membrane circumferential direction (sᵣ). A frame enclosing each pore is created using Weaverbird's Picture Frame function, with an offset thickness stipulated by the user. The offset thickness of the frame was set equal to half the pore separation distance to generate a pore with the desired diameter. After this step the mesh is thickened to desired capillary membrane thickness and smoothed using Weaverbird and Catmull-Clark functions.

To suspend the capillary membrane off the printing substrate, we implemented two scaffold support strategies: polygonal support and lattice-based support structures. The polygonal support method adds beams arranged in polygon shape around the capillary, connected via diagonal struts to a circular collar that wraps around the perimeter of the capillary. Users can define support spacing, polygon type, and rotational offset. Supports may be uniformly distributed or strategically placed at predicted field-of-view (FOV) transitions based on the TPL system's lens and slice settings (FIG. 1B). A Hilbert curve-based microcapillary illustrates this method, with lighter color support beams placed at FOV intersections (FIG. 1C). The design platform is scalable, easily accepting larger and more complicated 2D paths (FIG. 1D). Examples of 3D configurations using this support strategy are shown, including Hilbert curves (FIG. 1E) and double helices (FIG. 1F).

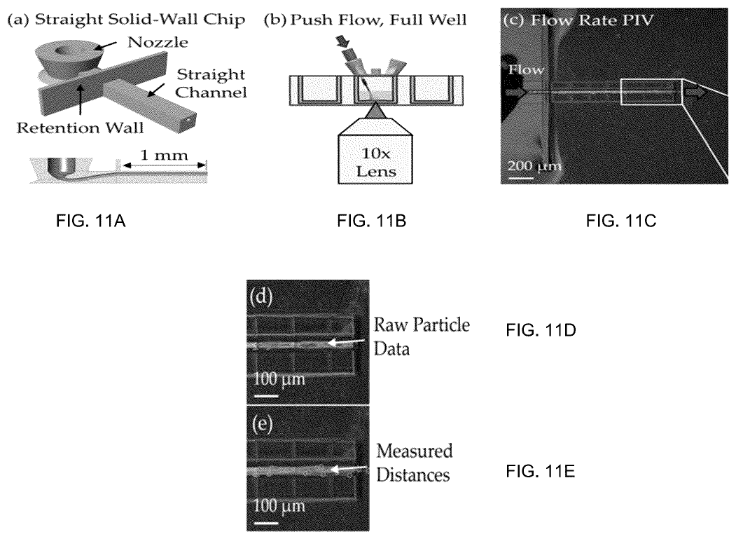

However, this approach relies on the connection at the bottom to the base and cannot fully support arbitrary 3D curves. To address this, we employed a lattice-based strategy using a voxelized bounding box generated via the Crystallon plugin (FIG. 1G), which ensures the connections among all unit cells at the box boundary. A truncated octahedron unit cell was selected for its high porosity and structural connectivity. Importantly, lattice beams intersecting the capillary volume were removed using the PointInBrep function, and the remaining struts were converted to solid pipe Brep objects with rounded caps, rendered in gold color. This support method not only enables fabrication of complex 3D geometries but also promotes 3D cellular integration. Examples using 3D Hilbert and double helix capillaries demonstrate the method's capabilities (FIGS. 1G and 11). This flexible approach facilitates rapid tuning of architectural complexity, microcapillary spacing, and spatial coverage for a range of in vitro tissue modeling applications.

TPL Materials and Resin Preparation: OrmoComp (Micro Resist Technology GmbH) and IP-PDMS (Nanoscribe GmbH) resins were used as received from the manufacturers. These materials provided rigid and elastomeric mechanical properties, respectively, and were used to fabricate scaffold regions requiring high geometric fidelity (OrmoComp) or flexible, tissue-mimicking characteristics (IP-PDMS).

PEGDA with a molecular weight of 700 kDa (PEGDA 700, Sigma-Aldrich) was selected as a moderately stiff material to balance geometric stability and biological relevance. PEGDA 700 was filtered through a MEHQ column filter to remove polymerization inhibitors, then mixed with 1% w/v Irgacure 819 photoinitiator (Sigma-Aldrich). The mixture was vortexed at 2500 rpm for 1 minute to ensure homogeneity. Air bubbles were removed by vacuum degassing prior to TPL printing.

By combining rigid, intermediate, and elastomeric resins, this material system allowed tuning of scaffold mechanical properties and supported fabrication of perfusable microcapillary scaffolds for versatile cell culture applications.

TPL Fabrication of Microcapillary Designs: Microcapillary scaffolds were fabricated using TPL to achieve high-resolution, geometrically precise structures suitable for integration into microfluidic platforms. Scaffold models, including capillaries, support lattices, and base geometries, were designed in Rhinoceros 3D and exported as STL files. Test scaffolds were initially printed with a range of lumen diameters (40-80 μm), and a 40 μm diameter capillary chip based on two stacked Hilbert curves was used for structure testing and flow validation. A flat base was added below the scaffold to help ensure adhesion in aqueous conditions.

All scaffold component models were converted to mesh format, and mesh density was reduced by 95% using the ReduceMesh command in Rhino 7 to minimize file size for efficient slicing in DeScribe software (Nanoscribe GmbH). The models were then sliced in DeScribe using optimized parameters: a slice thickness of 1.0 μm, hatch distance of 0.2 μm, and six contour lines. Solid infill was applied to ensure structural integrity, and stitching across adjacent fields of view (FOVs) was controlled using a 15° stitching angle and 5 μm overlap to minimize discontinuities.

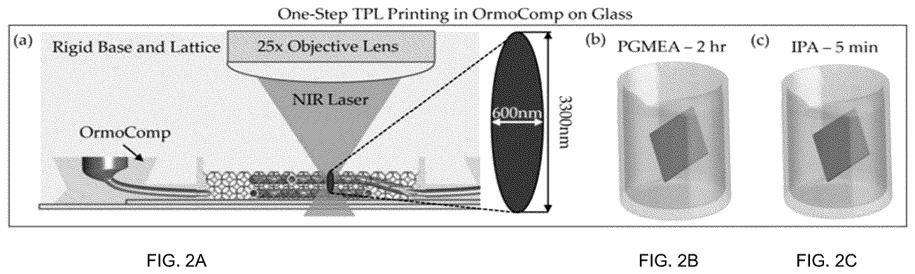

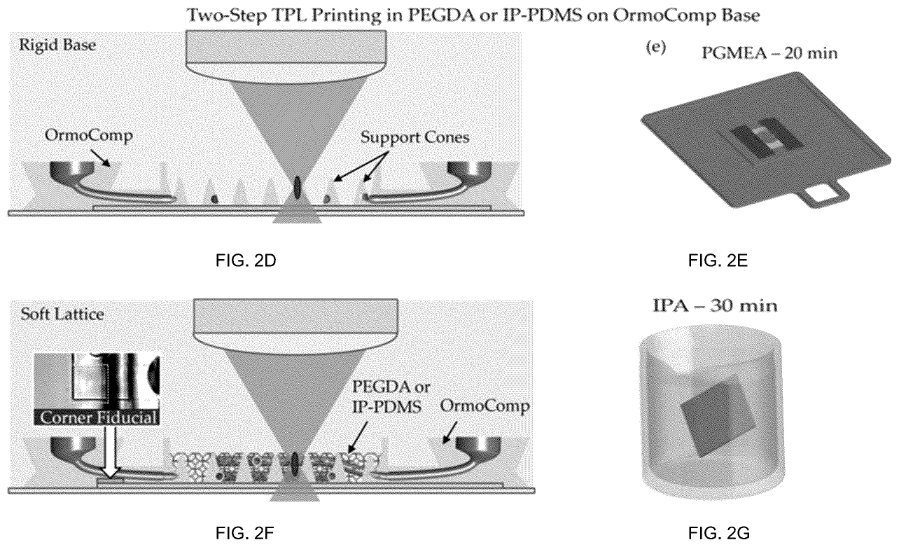

Fabrication was performed on a Nanoscribe Photonic Professional GT+ system equipped with a 25×, NA 0.8 immersion objective, operating in Dip-in Laser Lithography (DiLL) mode with a 780 nm femtosecond laser. A schematic of the DILL printing process is shown in FIG. 2A. Coverslips (12 mm diameter, No. 2 thickness) were cleaned sequentially with acetone, isopropanol, and deionized water, followed by oxygen plasma treatment. To promote scaffold adhesion, Ormoprime was spin-coated onto the coverslips at 4000 rpm for 1 minute and baked at 150° C. for 5 minutes. The prepared coverslips were taped on all sides onto indium tin oxide (ITO)-coated glass slides for mounting. A focal offset of approximately 200 μm (glass slide thickness) was applied to target the focal plane ~8 μm inside the coverslip, ensuring uniform adhesion and printing consistency across the sample.

In TPL, a near-infrared (NIR) femtosecond laser is tightly focused to induce two-photon absorption in the resist. At the focal point, the simultaneous absorption of two photons initiates local crosslinking, forming a hardened voxel with a diameter of approximately 600 nm in the short axis and 3300 nm in the long axis [30]. Printing parameters were adjusted based on the material properties. For OrmoComp scaffolds, printing was conducted using a laser power of 40 mW and a scan speed of 80 mm/s. After printing, the structures were developed in a PGMEA bath for 2 hours, followed by a 5-minute rinse in isopropanol (IPA) (FIGS. 2B-2C).

For soft materials, including PEGDA 700 and IP-PDMS, a dual-material develop-in-place workflow was implemented to prevent collapse during printing and post-processing. Initially, OrmoComp base structures with integrated nozzle ports were printed directly onto the coverslip and developed in PGMEA for 20 minutes, rinsed with IPA, and dried with nitrogen (FIGS. 2D-2E). A large droplet of OrmoComp was required to avoid bubble forming in the second layer of the 3D printed structure.

Next, PEGDA 700 or IP-PDMS resin was manually dispensed onto the developed OrmoComp base. Air bubbles were carefully removed with forceps to avoid potential printing defects. The sample was reloaded into the Nanoscribe system, and the objective was manually realigned using recorded focal height offsets. The alignment was further fine-tuned by printing fiducial marks at the corner of the scaffold to ensure accurate overlay (inset of FIG. 2F). Capillary networks and lattice supports were then printed directly on the OrmoComp base, which contains cone structures strategically printed on the base to provide distributed support of the soft scaffolds. PEGDA 700 was printed using a laser power of 50 mW and a scan speed of 74 mm/s, while IP-PDMS was printed using a laser power of 40 mW and a scan speed of 50 mm/s.

Upon completion, scaffolds printed in PEGDA and IP-PDMS were developed in IPA for 30 minutes to remove uncrosslinked resin and air dried (FIG. 2G). This dual-material fabrication process successfully integrated rigid and soft polymers into single scaffolds, enabling complex geometries with enhanced mechanical robustness while supporting soft capillary networks that resisted collapse during solvent development.

Microfluidic Interface: To establish perfusion through the TPL-printed microcapillary scaffolds, a modular microfluidic interface was engineered. This system utilized polyolefin heat-shrink tubing (McMaster-Carr) and 22-gauge hypodermic needles (Careach) to provide a compact, scalable, and cost-effective alternative to bulky commercial connectors that are often incompatible with delicate microscale architectures.

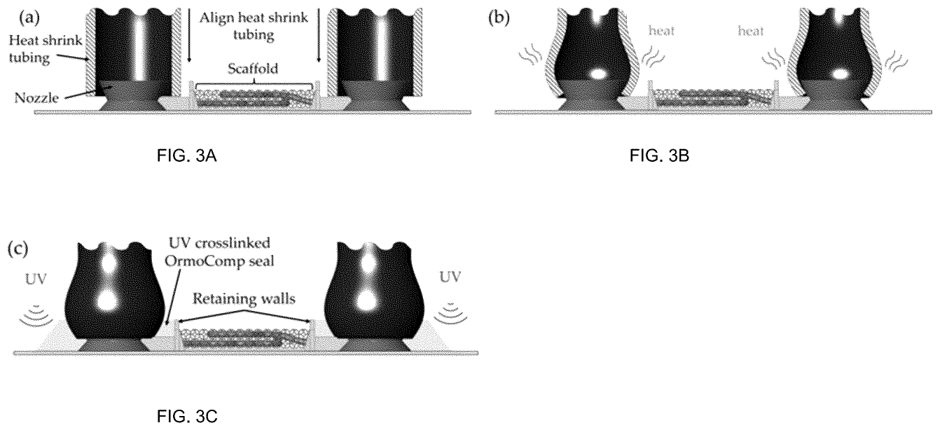

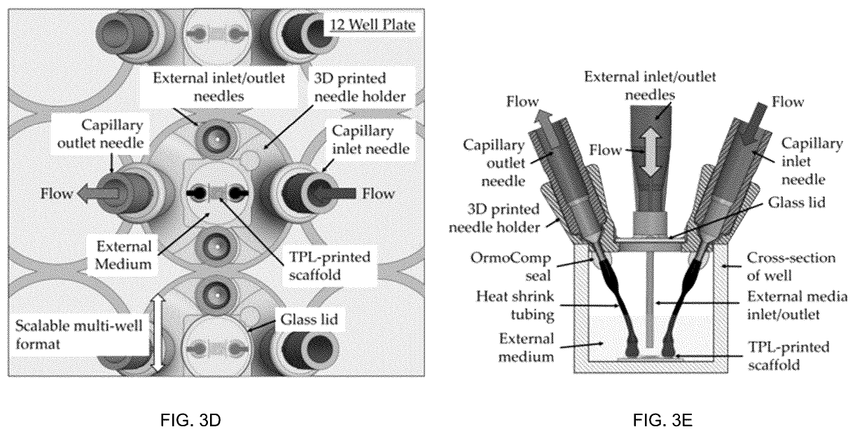

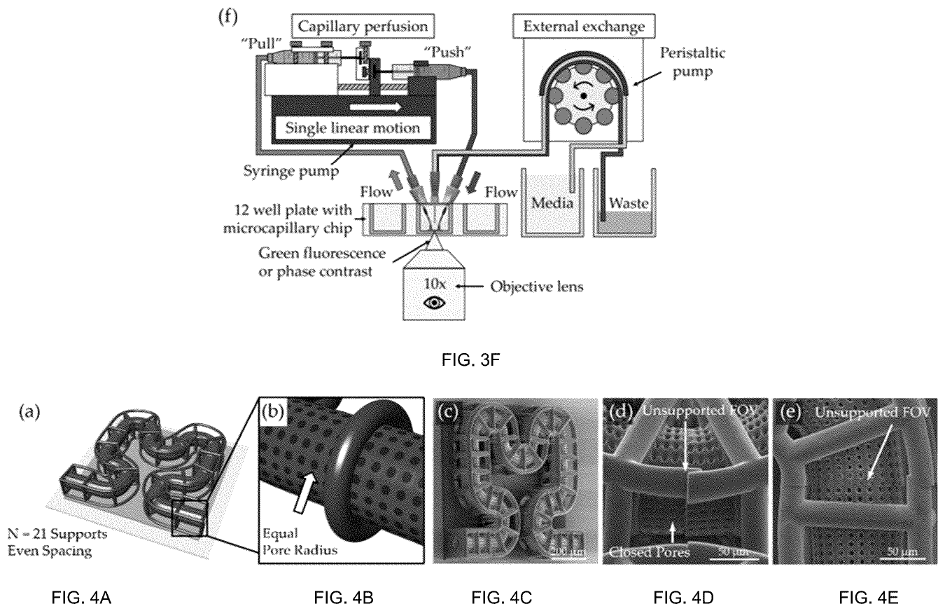

The assembly process is shown in FIGS. 3A-3C. Vertical nozzle ports were designed directly into the OrmoComp scaffold base and were optimized to mate with 0.020-inch inner diameter heat-shrink tubing (McMaster-Carr) (FIG. 3A). Following scaffold development, the tubing was manually fitted onto the nozzle ports and locally heated using a soldering iron for 1-2 seconds. This heating step caused the tubing to contract, creating a tight, press-fit seal against the nozzle surfaces (FIG. 3B). To further secure the connection and prevent leakage, OrmoComp resin was applied around the tubing at the junction. A retaining wall was used to prevent resin from spreading into the microvasculature. The resin was cured under ultraviolet (UV) light at 100 mW/cm² for 1 minute (FIG. 3C).

The entire assembly was then secured to the bottom of a standard 12-well plate using a small drop of OrmoComp resin, which was UV-cured to anchor the chip firmly in place. A custom 3D-printed holder was affixed to the top of the well using UV-cured OrmoComp. The top-down view is shown in FIG. 3D, and the side view of single well is shown in FIG. 3E. This holder included ports for two blunt 26-gauge hypodermic needles (Careach, 0.5-inch length), enabling external media exchange during experiments.

Flow system integration was achieved by inserting blunt 22-gauge hypodermic needles into the exposed ends of the heat-shrink tubing. After insertion, the heat-shrink tubing was locally heated with a soldering iron to shrink tightly around the needles, ensuring robust mechanical engagement. OrmoComp resin was then applied around the needle-tubing junction and UV-cured (100 mW/cm², 1 minute) to complete a leak-free seal.

The chip was covered with a removable 12 mm diameter glass lid to fully enclose the chamber and maintain sterility. For perfusion experiments, the assembled device was connected via flexible tubing to a syringe pump operated in a push-pull configuration, allowing controlled, bidirectional flow through the scaffold (FIG. 3F). This setup minimized pressure fluctuations and ensured gentle, continuous perfusion compatible with neurovascular models. An in-situ imaging system (Etaluma LS560 microscope equipped with a custom 3-axis stage) was used to monitor perfusion in real time without disrupting incubator conditions (FIG. 3E).

Sterilization and Scaffold Presoak: To ensure biocompatibility and minimize cytotoxic effects from fabrication residues, all microfluidic chip components underwent rigorous sterilization and presoak protocol prior to biological testing. This two-stage process was designed to remove leachable contaminants and condition scaffold surfaces for optimal cell interaction.

All chip materials, including TPL-printed scaffolds (OrmoComp, PEGDA 700, and IP-PDMS), heat-shrink tubing, 3D-printed parts (Siraya Tech Blu, Anycubic Transparent Green, and PLA), and tissue culture plastics, were sterilized after assembly. Microfluidic chip assemblies were first rinsed with 70% ethanol for two minutes. After air-drying in a laminar flow hood for 30 minutes, the chips were exposed to germicidal ultraviolet (UV) light for 1.5 hours.

Following sterilization, a prolonged presoak process was implemented to further reduce potential cytotoxic leachates, such as residual photoinitiators and unreacted monomers. During the initial two days (Days 0-1), chips were rinsed at one-hour intervals for three hours with phosphate-buffered saline (PBS) to remove soluble impurities. From Days 2 to 6, chips were washed daily and incubated in complete neural growth medium to allow surface equilibration and passivation. This presoak stage was critical for minimizing acute cytotoxic responses upon subsequent cell seeding.

The neural growth medium used during presoak was formulated to support neural stem cell health. It consisted of basal medium (Vesta Biotherapeutics, formerly Phoenixsongs Biologicals) supplemented with Neural StemCell Growth Base Supplement, laminin, basic fibroblast growth factor (bFGF), epidermal growth factor (EGF), and a proprietary neural culture factor. Gentamicin (37.5 mg/L) was included to prevent microbial contamination during extended incubation. All media components were sterile filtered using a 0.22 μm membrane and stored at 4° C. prior to use.

Surface Coating: To enhance cellular adhesion and prepare scaffold surfaces for neural cell culture, a sequential coating protocol using poly-D-lysine and laminin was applied. This approach improved scaffold biocompatibility and mimicked essential extracellular matrix features.

Scaffolds and controls (tissue culture plastic) were first incubated in a 5 μg/mL solution of poly-D-lysine for 10 minutes at room temperature. This cationic polymer increases surface charge and promotes initial cell adhesion on synthetic and hydrogel-based materials. Following incubation, samples were rinsed three times with sterile, cell culture-grade water to remove unbound poly-D-lysine and ensure uniform surface coating.

Next, laminin was applied at a concentration of 10 μg/mL and incubated for 2 hours at room temperature. As a key extracellular matrix protein, laminin supports neuronal attachment, differentiation, and survival, making it critical for creating a cell-compatible microenvironment. After incubation, excess laminin was aspirated and samples were gently rinsed with sterile water to remove residual solution. Culture medium was then added to the wells to maintain hydration and prepare samples for cell seeding. To stabilize the coating and equilibrate the scaffolds to culture conditions, coated samples were placed in a humidified incubator for 1 hour prior to cell plating.

Cytotoxicity Assay: The cytocompatibility of microcapillary scaffolds and associated microfluidic chip materials was evaluated using a Live/Dead® viability assay (Thermo Fischer Scientific) with human hippocampal-derived neural stem cells (hNSCs; HIP-009, Vesta Biotherapeutics). These cells, chosen for their sensitivity to material-induced toxicity and relevance to neurobiological studies, provided an effective model for assessing material suitability for brain-on-chip applications.

Cells were seeded directly onto scaffold surfaces and control substrates immediately after thawing from cryopreservation at a density of 4.5×10⁵ cells per well. Non-adherent cells and debris were removed by media exchange on Day 1 to ensure uniform attachment. Cultures were then maintained without further media changes until Day 3 under static conditions.

At the conclusion of the culture period, viability was assessed using the Live/Dead assay, which differentiates live and dead cells by fluorescence. Calcein AM (2 μM, Ex: 484 nm, Em: 517 nm) labeled viable cells green, while ethidium homodimer-1 (4 μM, Ex: 528 nm, Em: 617 nm) stained non-viable nuclei red. Samples were incubated in staining solution for 20 minutes at 37° C. before imaging.

Fluorescence imaging for cytotoxicity assay and scaffold material fluorescence analysis was performed on an Olympus IX-70 inverted microscope equipped with filter cubes: U-MNUA2 for blue (Ex: 365 nm, Em: 440 nm), U-MNIBA3 for green (Ex: 480 nm, Em: 530 nm), and U-MRFPHQ for red (Ex: 545 nm, Em: 597 nm). Images were captured using a CoolSnap K4 camera (Photometrics) controlled by MicroManager software. Exposure times were standardized to 1000 ms across all channels to ensure consistency.

Quantitative analysis was conducted by imaging three independent regions per sample at 4× magnification, yielding nine images per material group. Images were processed using a custom MATLAB script for automated segmentation and initial cell counting, followed by manual correction with a secondary script to verify accuracy. In total, viability calculations were based on approximately 116,000 cells.

PEGDA Mechanical Property Characterization: While mechanical data for OrmoComp and IP-PDMS produced by TPL are available in the literature, equivalent characterization for PEGDA 700 formulated with 1% Irgacure 819 photoinitiator had not been reported. As this formulation was specifically utilized for nanoprinting in this study, direct measurement of its mechanical properties was necessary.

Test specimens were printed using TPL as square prisms (200 μm×200 μm×10 μm) onto methacrylate-treated glass coverslips (3 hours in 3-(Trimethoxysilyl) propyl methacrylate) to ensure adhesion during testing. Nanoindentation was conducted under ambient conditions using a Hysitron TriboIndenter equipped with a spheroconical diamond probe (5 μm tip radius). A linear load-unload rate of 10 μN/s was applied up to a peak load of 250 μN, with a 5-second hold at maximum load.

The Young's modulus and hardness of PEGDA 700 were calculated based on the nanoindentation load-displacement curves using the Oliver-Pharr method. Reported values reflect the mean±standard deviation from three indentations.

Geometry Registration Analysis: A geometry registration analysis was performed to quantitatively assess scaffold fidelity by comparing fabricated structures to their original digital designs. Deviations were expected to arise from two sources: inaccuracies in the printing process and structural deformation during post-fabrication steps (such as solvent development and drying). As imaging was performed only after development, the measured deviations reflect the combined contributions of both factors.

Binary reference masks representing the intended geometries were generated from the original design files. Top-down renders of scaffold models were exported from Rhinoceros 3D and processed in ImageJ to produce binarized black-and-white target images. These served as the reference standard for assessing print fidelity.

Experimental masks were created from scanning electron microscopy (SEM) images of the printed scaffolds. SEM images were first processed to enhance feature visibility, including a global contrast enhancement of 30%. For regions with faint features, particularly at the image edges, localized adjustments were manually applied to improve clarity. The processed images were then binarized to isolate capillary membrane and lattice structures.

To address areas where thresholding alone did not sufficiently capture scaffold features, edge detection using the “Find Edges” function in ImageJ was combined with manual editing to fill in missing regions. Cone support structures were digitally subtracted to ensure that only relevant scaffold features remained for analysis.

Target and experimental masks were then merged using color channels to generate composite registration maps. The target design was assigned to the green channel and the experimental print to the red channel. Therefore, in the resulting maps, yellow indicated overlapping features, green indicated missing scaffold material, red indicated deformed printed material, and black indicated void regions. The overlapping areas were analyzed. This registration method allowed systematic evaluation of printing fidelity, capturing combined deviations resulting from both printing imprecision and post-processing deformation.

Diffusion and Particle Flow Testing: Functional testing of the assembled chips was conducted using fluorescein-labeled Dextran and 1 μm fluorescent polystyrene beads. For diffusion experiments, Dextran in phosphate-buffered saline (PBS) was perfused through the capillary scaffolds using a syringe pump (Pump Systems Inc.) configured with an auxiliary syringe holder to enable push-pull flow (FIG. 3F). A peristaltic pump (Ismatec) was used to circulate medium in the external chamber surrounding the scaffold. The syringe pump was operated at a flow rate of 1 μL/min, and diffusion across the capillary membrane was imaged in situ using an Etaluma LS560 microscope. The camera exposure time was set to 5.1 ms to match calibration conditions for Dextran fluorescence.

To assess flow speed, PIV was performed by perfusing 1 μm green fluorescent beads through a 1 mm-long solid-walled channel with a 40 μm diameter-equivalent to the lumen geometry of the scaffold. This open-channel configuration was used to avoid clogging observed in earlier scaffold-based attempts. Beads were suspended in water and driven through the channel at 10 nL/min using the same syringe pump in unidirectional push mode. Timelapse videos were acquired and analyzed using a custom MATLAB script to extract bead trajectories and calculate flow velocity.

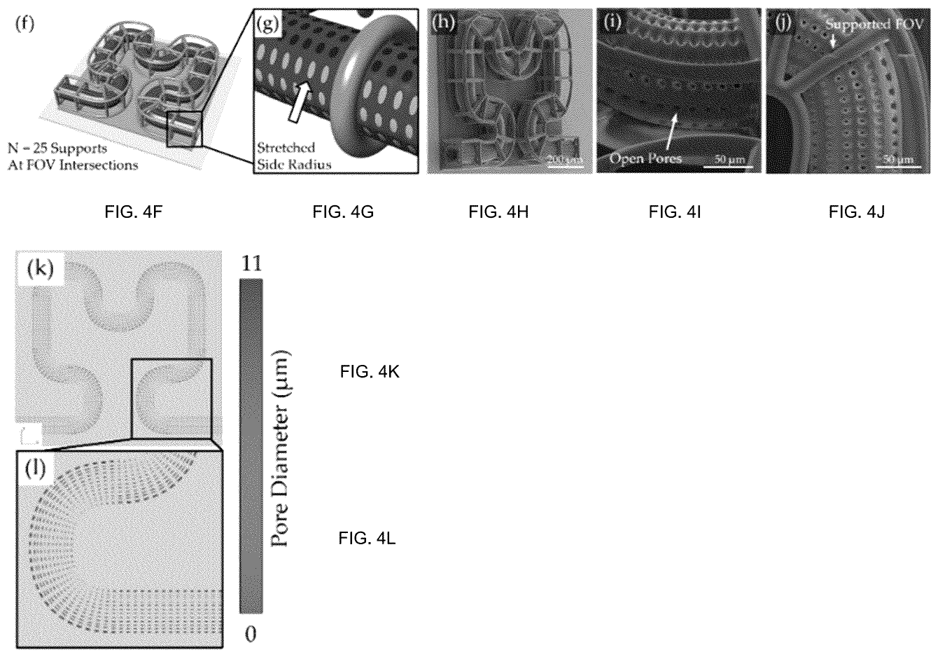

Results: Scaffold Fabrication with Polygonal and Lattice Support Strategies: Support strategies for microcapillary fabrication were evaluated using a planar Hilbert curve scaffold design. This model, shown in FIG. 4a, featured a lumen diameter of 80 μm, pore diameter of 5 μm (FIG. 4B), pore spacing of 5 μm, membrane thickness of 15 μm, and polygonal support rings with a 20 μm diameter positioned along the capillary path. Initial fabrication trials using this configuration (FIGS. 4C-4E) revealed critical limitations. Although the overall scaffold geometry was achieved, closed pores were observed along the lateral sides of the capillary membrane, and significant misalignment occurred at FOV intersections. These issues were most pronounced in unsupported regions where capillary sections were suspended without reinforcement.

To address these problems, several design modifications were implemented. The pore was elongated in the circumferential direction by approximately 1.5× to keep the pores from closing. In addition, the support beams were repositioned to align with predicted FOV intersections (FIGS. 4F-4G). By making these changes, the structure was able to be redesigned with reduced beam diameters of 15 μm to reduce material usage and increased pore spacing to 8 μm separation for added stability. These adjustments improved scaffold fidelity. SEM imaging confirmed preservation of pore openings and reduced stitching misalignment (FIGS. 4H-4J). However, uniformity of pore sizes remains a challenge. Analysis of designed pore diameters along the scaffold path (FIGS. 4K-4L) showed significant variability, with pores constricting on interior curves and widening on exterior curves. Additionally, the polygonal support structures rely on direct connection to the base, limiting the vasculature format to 2D curves parallel to the surface.

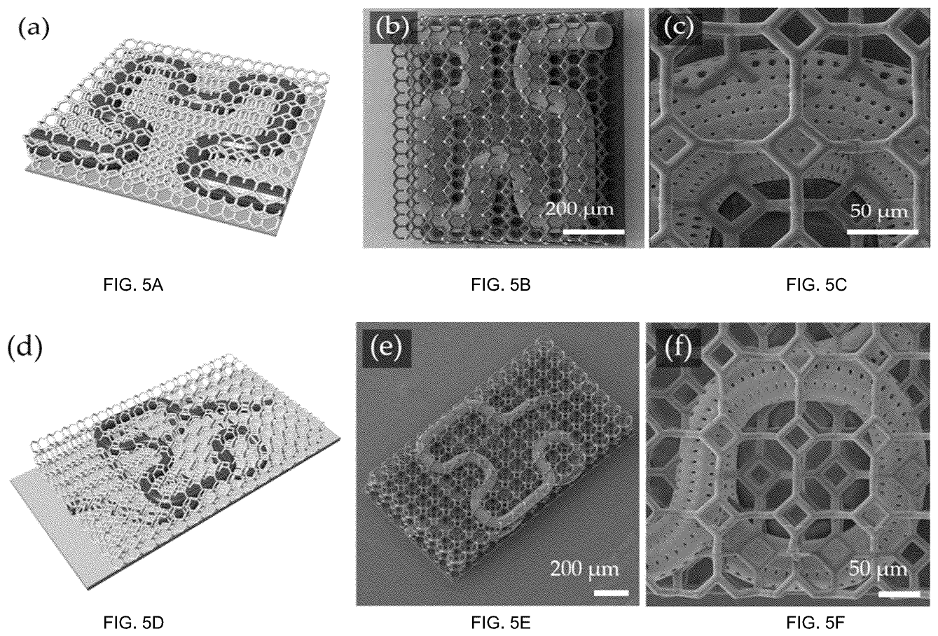

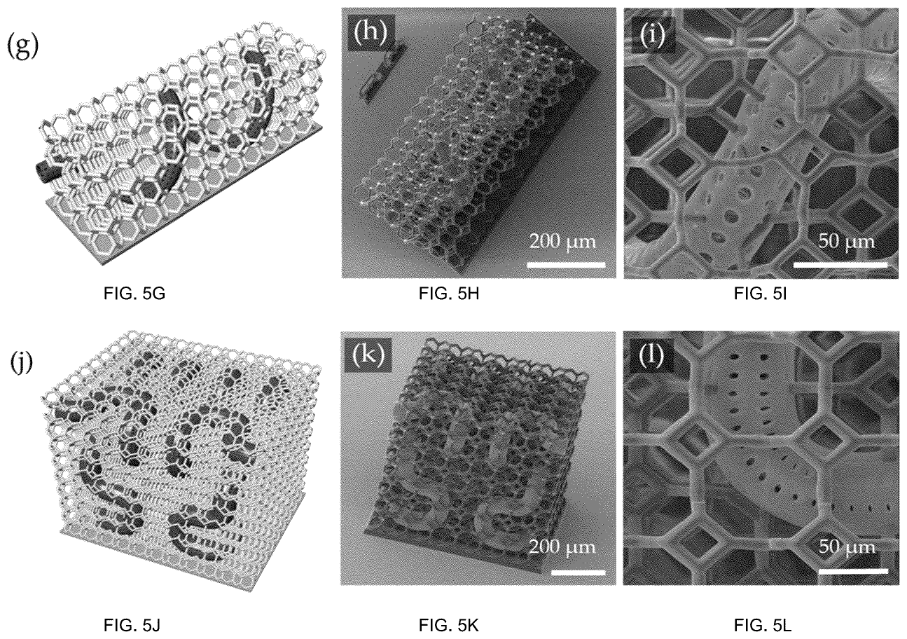

To overcome these constraints, a lattice support strategy was introduced to provide continuous reinforcement throughout the scaffold volume. A lattice-supported version of the planar Hilbert curve scaffold was fabricated using the same overall geometric parameters (FIGS. 5A-5C). It was shown that, due to higher support density, the support beam diameter could be reduced to 10 μm without affecting print fidelity. Colorized SEM micrographs, used to improve visualization of scaffold geometry, confirmed robust 3D support and substantially improved structural stability, particularly at FOV intersections where deformation had previously been problematic.

The versatility of lattice supports was further demonstrated through fabrication of additional capillary scaffold designs. These included a vertically stacked two-layer Hilbert curve with a lumen diameter of 50 μm (FIGS. 5D-5F), a 3D helical scaffold with a 40 μm lumen diameter (FIGS. 5G-5I), and a second-order 3D Hilbert curve with a 50 μm lumen diameter (FIGS. 5J-5L). All scaffolds were printed using OrmoComp and exhibited no major defects, confirming that the lattice support strategy enabled reliable fabrication of complex three-dimensional microvascular architectures.

Multi-material Scaffold Fabrication, Characterization, and Geometric Fidelity: Following validation of scaffold designs using OrmoComp, multi-material printing trials were conducted to investigate the feasibility of fabricating microcapillary scaffolds from materials with differing mechanical properties. OrmoComp, PEGDA 700, and IP-PDMS were selected to represent rigid, hydrogel, and elastomeric classes, respectively. These materials span a wide Young's modulus spectrum and address different demands for rigidity, elasticity, and biomimicry. While OrmoComp and IP-PDMS had previously reported mechanical properties under relevant processing conditions (~1.27 GPa and ~17.5 MPa Young's modulus, respectively).

PEGDA 700 formulated with 1% Irgacure 819 photoinitiator had not been characterized. Thus, direct mechanical testing was required.

PEGDA 700 square prisms (200×200×10 μm) were printed onto methacrylate-treated glass coverslips and tested using nanoindentation. The measured Young's modulus in dry condition was 30.7±1.2 MPa, with a hardness of 8.3±0.4 MPa. These values positioned PEGDA 700 between OrmoComp and IP-PDMS in elasticity, confirming its moderate rigidity and potential for scaffold fabrication. However, as expected, PEGDA 700 and IP-PDMS posed more significant challenges during fabrication due to their lower moduli and higher propensity for deformation during solvent development and drying.

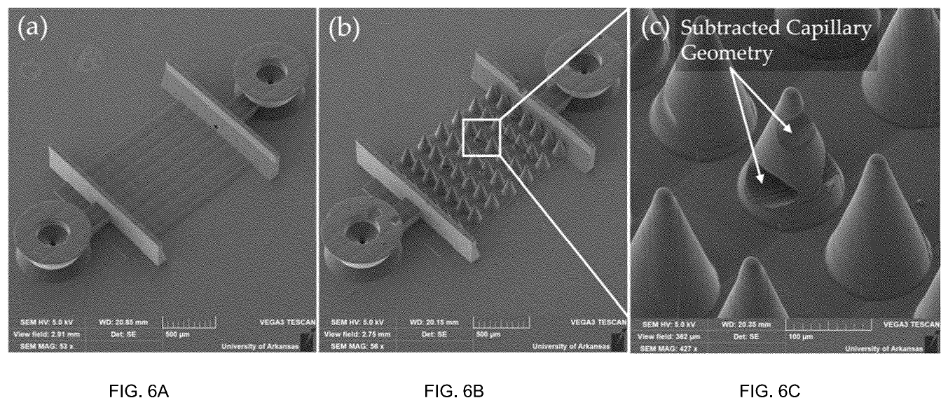

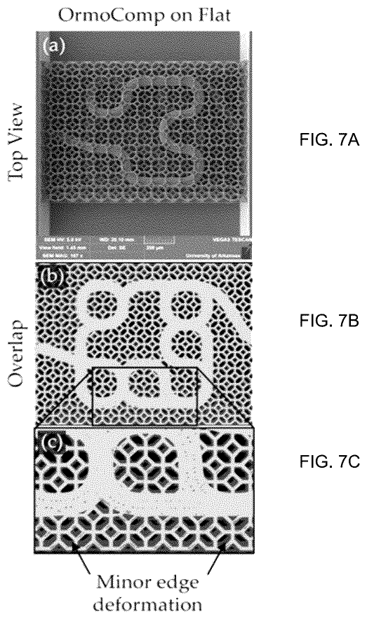

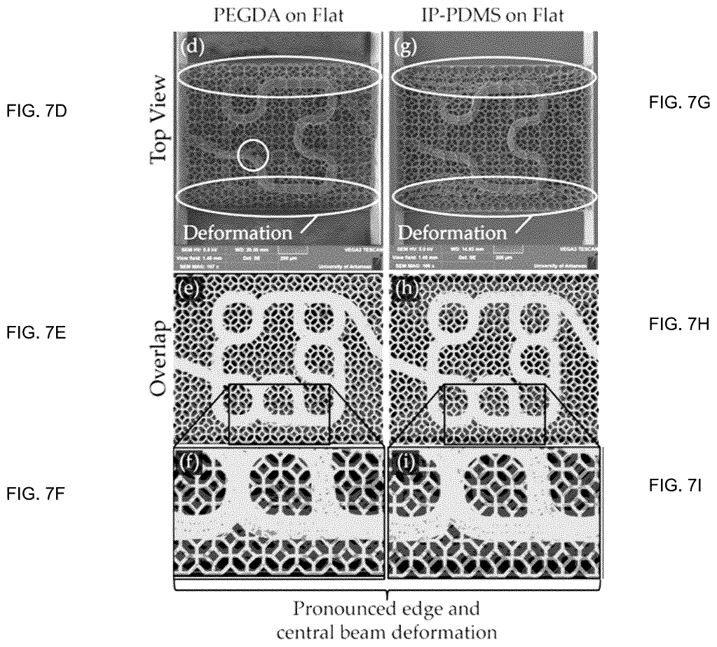

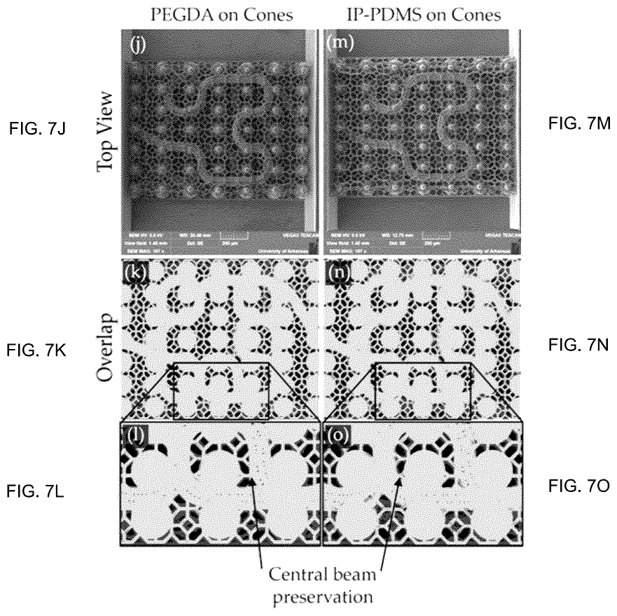

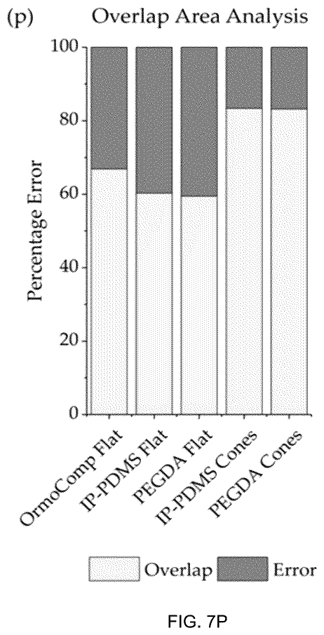

To enable rigorous comparative analysis, scaffolds using the layered Hilbert-curve chip design shown in FIG. 5D with a 40 μm lumen diameter, 5 μm pore diameter, 8 μm pore spacing, 10 μm membrane thickness, and 10 μm lattice beam diameters were fabricated across all materials. OrmoComp scaffolds were first printed directly on flat bases (FIG. 6A) and demonstrated good printing fidelity with minor edge deformation (FIG. 7A). In contrast, PEGDA 700 and IP-PDMS scaffolds printed on flat bases (FIGS. 7D and 7G) exhibited pronounced collapse, especially at curved regions and scaffold edges.

To mitigate these issues, mesoscale cone support structures were employed. The cones surface, shown in FIG. 6B, enables geometry to be supported in the vertical direction. These OrmoComp-printed cones featured minimum taper angles of 15°, which were determined to be necessary to prevent printing errors caused by vertical and overhanging geometry blocking the laser path. The capillary path was subtracted from the cones' geometry (FIG. 6C) to ensure the cones did not block perfusion.

The cones provided critical support to stabilize soft materials during both printing and development. By printing onto cone-supported OrmoComp bases, both PEGDA 700 and IP-PDMS scaffolds displayed substantially improved outcomes (FIGS. 7J and 7M). The conical supports minimized deformation in the central area, though defects were still observed near the edges.

Geometric registration maps were generated to visualize scaffold deviations. Color-coded overlays, shown in FIGS. 7B, 7E, 7H, 7K and 7N, revealed key deformation patterns. In color versions (not provided), yellow indicated correct overlap, green denoted missing material, red highlighted deformed geometry, and black represented void regions. Notably, excess material was predominantly located at scaffold edges, where deformation during drying was most pronounced. OrmoComp showed the highest fabrication accuracy on the flat base. The cone supports helped improve the outcomes of the IP-PDMS and PEGDA printing strategies, resulting in even better fidelity than using OrmoComp alone, underscoring the importance of appropriate support methods for softer material architectures.

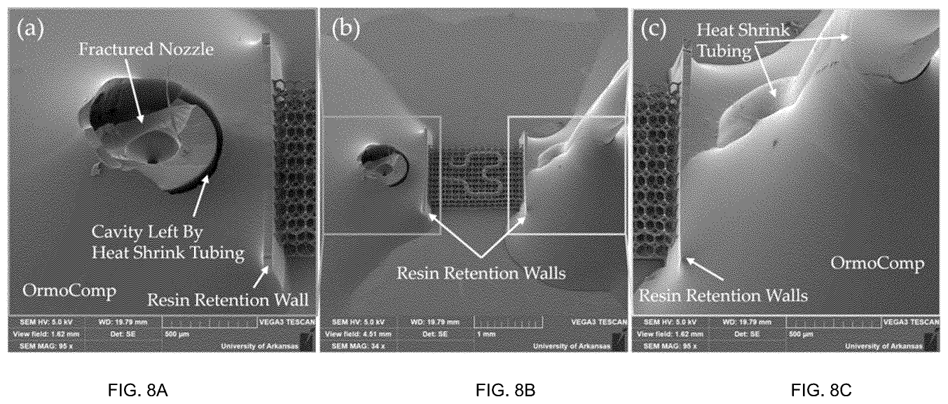

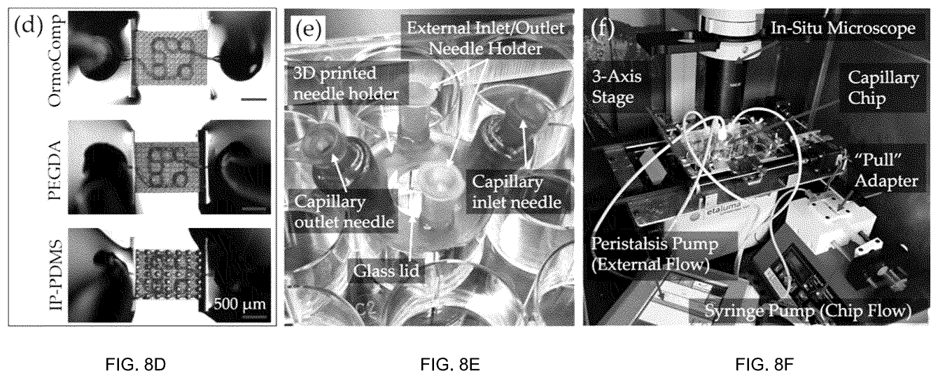

Microfluidic Chip Sealing and Integration for Flow Testing: The integrity of the microfluidic sealing strategy was evaluated through SEM and integration into a flow test setup is shown (FIGS. 8A-8F). During disassembly tests, removal of the heat-shrink tubing fractured the printed nozzle structure itself (FIGS. 8A and 8B). This fracture was most likely caused by compressive forces exerted between the tubing and the nozzle when removing heat shrink tube, indicating that a mechanically durable and leak-resistant bond had been formed. Importantly, examination of nozzle cross-sections (FIGS. 8B and 8C) confirmed that the OrmoComp resin seal was well confined within retention walls and did not extend into the scaffold lattice region, preserving open perfusion pathways essential for subsequent flow experiments.

Assembly of the completed microfluidic chip is shown in FIGS. 8D and 8E. The design utilized hypodermic needles as inlet and outlet inserted into heat-shrink tubing. The needles were UV-cured securely inside the 3D printed needle holder using OrmoComp, enabling capillary perfusion ports and external ports for media exchange outside the capillary. The chip was housed within a standard 12-well plate with a removable glass lid (FIG. 8E). This configuration maintained the chip sterility while enabling optical imaging access.

To integrate the chip into flow testing, it was connected to a syringe pump configured in push-pull mode for continuous flow and a peristaltic pump for external media exchange within the well volume. Mounted on an Etaluma LS560 microscope equipped with a custom 3-axis stage, the assembled chip allowed in situ visualization of perfusion pathways during operation, without disrupting incubator conditions (FIG. 8F).

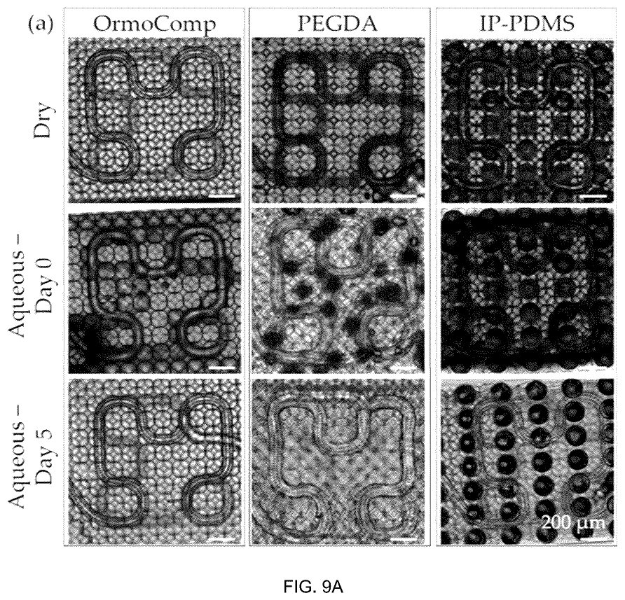

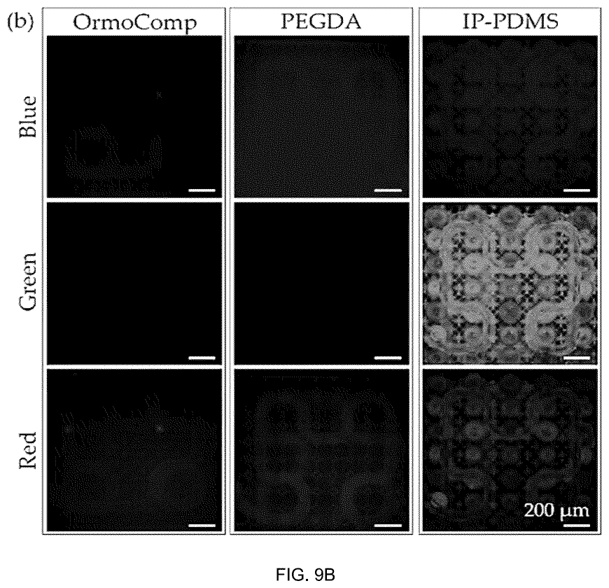

Evaluation of Scaffold Dimensional Stability, Diffusion Transport, and Perfusion Performance: Following chip fabrication and integration into the perfusion setup, the system was evaluated for integrity under cell culture-like conditions. The first assessment focused on scaffold integrity after immersion in aqueous media. Brightfield imaging of dry and hydrated scaffolds revealed distinct responses across materials (FIG. 9A). OrmoComp and IP-PDMS scaffolds demonstrated excellent dimensional stability, maintaining their original geometries without visible deformation after immersion. In contrast, PEGDA 700 scaffolds exhibited visible distortion of both capillary membranes and lattice supports. Notably, this deformation proved largely reversible. After five days of continuous incubation, PEGDA 700 scaffolds gradually returned to near-original geometry, suggesting that the swelling was driven primarily by heterogeneous medium uptake across the lattice. Additionally, air bubbles initially trapped within the lumen of all scaffold types were passively released during this equilibration period. IP-PDMS retaining the greatest amount of air initially, while PEGDA and OrmoComp trapped relatively less air volume.

Next, scaffold autofluorescence was evaluated, as optical clarity and low background signal are essential for fluorescence-based biological imaging. Dry scaffolds were imaged using standard blue, green, and red filter cubes (FIG. 9B). OrmoComp exhibited the lowest autofluorescence across all channels, confirming its suitability for high-resolution fluorescence imaging. PEGDA 700 scaffolds displayed intense autofluorescence in the blue channel, while IP-PDMS showed moderate autofluorescence predominantly in the green channel with noticeable autofluorescence also seen in blue and red channels. These observations underscore the importance of careful fluorophore selection when performing immunochemical staining or live imaging, particularly for scaffolds fabricated from PEGDA 700 and IP-PDMS.

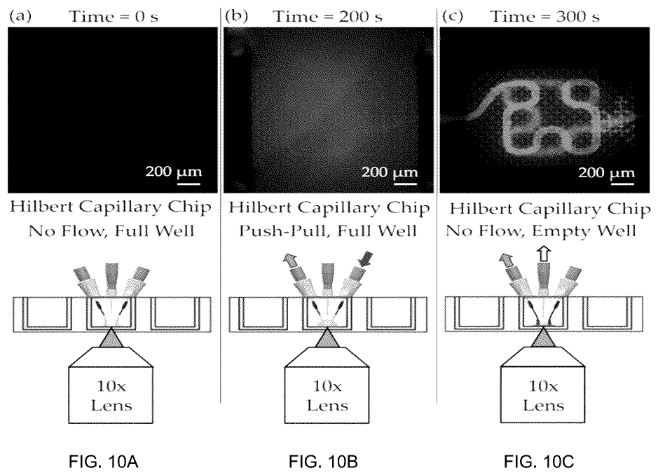

Molecular transport was then assessed using fluorescein-labeled Dextran to validate diffusion across the capillary membrane (FIGS. 10A-10C). Schematics of each testing condition are shown at the bottom row of the figure. An initial baseline image prior to flow confirmed the absence of background fluorescence (FIG. 10A). After 200 seconds of perfusion, Dextran diffused from within the capillary lumen into the surrounding medium, as indicated by increased fluorescence intensity outside the membrane (FIG. 10B). Subsequently, the Dextran was withdrawn through the outlet port using the push-pull flow configuration and the outlet for the external media. During this phase, a reverse concentration gradient caused Dextran to be reabsorbed into the capillary lumen (FIG. 10C, green region), demonstrating the scaffold's ability to support bidirectional diffusion across the membrane.

Finally, the ability of the printed scaffolds to support perfusion at biologically relevant flow speeds was evaluated using fluorescent polystyrene beads (1 μm diameter). Initial flow measurements in the microcapillary scaffold were limited by particle clogging. To address this, flow testing was performed using a straight microchannel (1 mm long, 40 μm in diameter) that replicated the lumen geometry of the scaffold (FIG. 11A). To minimize clogging risk, a simplified open channel configuration was used with one inlet and an open outlet leading into the well reservoir, as shown in the chip design schematic (FIG. 11A). Flow was driven using a syringe pump operating in a unidirectional push configuration (FIG. 11B).

Flow speed was measured using a custom MATLAB script for PIV. Video frames showing bead movement were extracted and compiled into a maximum intensity projection, i.e., a single image that captures the full trajectories of moving particles (FIG. 11C). Individual bead positions across frames were manually tracked using a graphical interface, and their displacements were used to calculate velocity. A raw frame showing tracked bead positions is shown in FIG. 11D, and the compiled trajectories from the full sequence are shown in FIG. 11E.

At a flow rate of 10 nL/min, the average velocity of 27 tracked particles was 1.83±0.56 mm/s. This falls within the physiological range of cerebral microvascular blood flow (0.1-9.4 mm/s), confirming that the fabricated microchannel and interfacing system are capable of supporting biologically relevant flow conditions.

hNSC Viability and Cytotoxicity Testing: To evaluate the biocompatibility of all materials integrated into the microvascular scaffold and microfluidic chip system, human hippocampal neural stem cells (HIP-009, Vesta Biotherapeutics) were used as a sensitive biological model. These cells were cultured directly on a range of substrates representing both scaffold fabrication materials and peripheral chip assembly components.

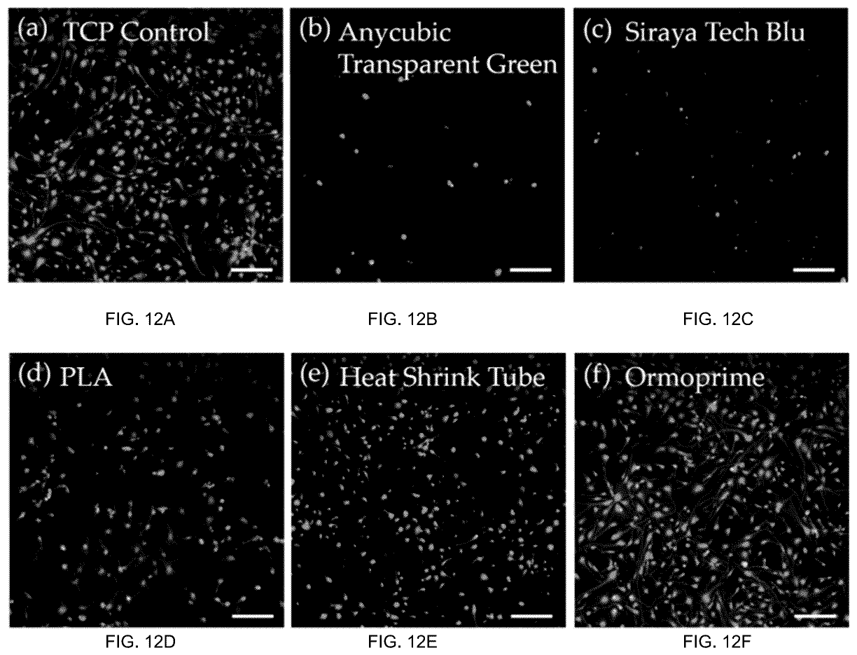

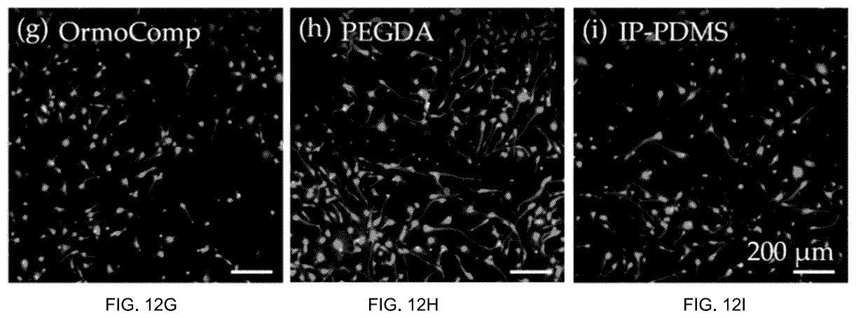

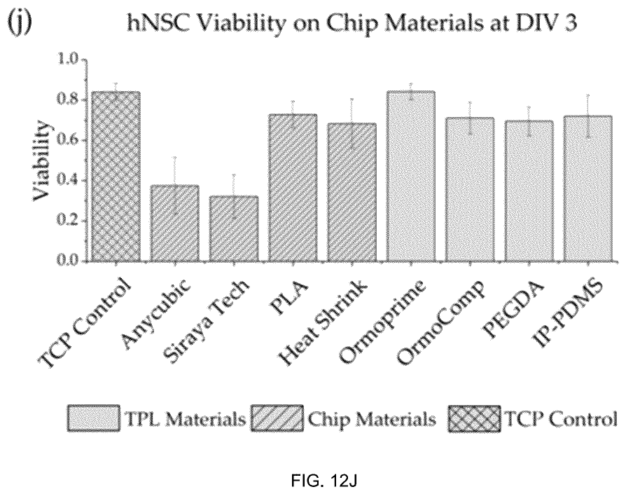

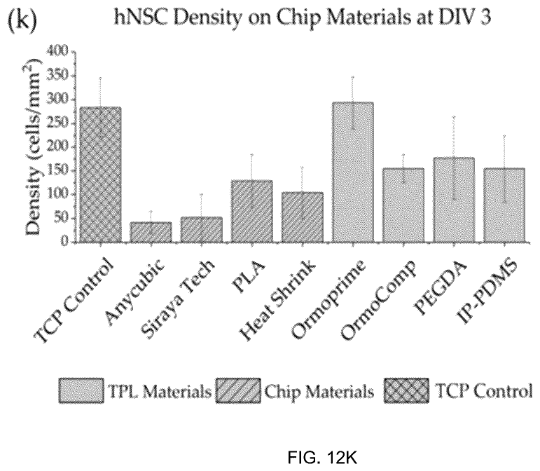

Representative fluorescence images from the Live/Dead viability assay, performed on day in vitro (DIV) 3, are shown in FIG. 12A-12I. The tested substrates were divided into two groups. The first group focused on the photopolymerizable resins used directly for scaffold fabrication, which are central to the microvascular chip system. These included Ormoprime adhesion promoter, OrmoComp organic-ceramic polymer, PEGDA 700 hydrogel, and IP-PDMS elastomer. As these materials form the functional scaffold architecture, confirming their compatibility with hNSCs was particularly critical.

The second group included chip assembly materials such as tissue culture plastic (TCP) as a positive control, heat shrink tubing, and three needle holder materials: polylactic acid (PLA) fabricated via fused deposition modeling (Creality Ender 3), and two photopolymer resins (Siraya Tech Blu and Anycubic Transparent Green) fabricated with an LCD 3D printer (Anycubic Photon Mono 4k). Notably, the LCD resin needle holder were placed on the tops of culture wells and did not directly contact the cell culture medium.

Quantitative viability analysis is summarized in FIG. 12J, with live cell densities shown in FIG. 12K. The results revealed suitable cytocompatibility exceeding 70% for OrmoComp, PEGDA 700, and IP-PDMS materials, which was also comparable to heat shrink tubing and PLA. Moreover, these materials supported consistent hNSC adhesion and spreading, confirming their suitability for neural culture applications. TCP and Ormoprime-treated surfaces exhibited the highest viability at around 84%.

In contrast, LCD resin needle holder demonstrated pronounced cytotoxicity. Both Siraya Tech Blu and Anycubic Transparent Green needle holder reduced cell viability to below 40%, and live cell density analysis revealed markedly lower cell attachment and spreading. These findings suggest the release of volatile compounds from LCD materials negatively impacted nearby cells, even in the absence of direct media contact. These printing materials should be avoided as microfluidic chip materials in future studies.

Discussion: This work advances the design and fabrication of microfluidic chips with embedded microvasculature scaffolds using TPL, addressing key challenges in generating physiologically relevant vascular structures. Compared to conventional CAD workflows, the mesh-based modeling strategy implemented in Rhinoceros 3D provided an efficient and flexible approach for creating complex scaffold geometries suitable for biological applications. By enabling rapid design iteration and integration of user-defined pore architectures and supports, this workflow streamlined the fabrication of customized vascular networks.

A persistent challenge in TPL fabrication of microcapillary scaffolds is maintaining open pore architectures, particularly along curved surfaces and scaffold edges. Due to the anisotropic voxel dimensions of TPL, pores facing lateral directions are especially prone to closure during printing. In this study, these limitations were addressed by elongating holes of the subsurfaces on the sides of the capillary membranes, effectively preserving side-facing pore openings and improving overall printing fidelity. Additionally, support strategies were critical for maintaining scaffold integrity throughout printing and post-processing. Polygonal support rings were sufficient for planar capillary designs but were inadequate for more complex geometries. Placement of lattice nodes at FOV stitching boundaries proved effective for reducing misalignment and ensuring seamless scaffold fabrication. In contrast, lattice supports, optimized for fully 3D scaffolds, provided distributed contact points that enhanced structural robustness and minimized deformation at the FOV intersections. The lattice supports were insufficient to provide structural stability for softer print materials such as PEGDA 700 and IP-PDMS. In contrast, the mesoscale cone supports, printed in OrmoComp, offered robust structural reinforcement, enabling successful fabrication of 3D capillary architectures even with soft materials. Both PEGDA and IP-PDMS demonstrated strong adhesion to the OrmoComp base, establishing a rigid interface that effectively anchors the soft TPL-printed scaffolds.

The ability to realize complex 3D capillary geometries using additive fabrication offers distinct advantages over existing subtractive and planar fabrication approaches. Previous studies relying on planar TPL geometries or subtractive methods, such as ablating hydrogels to create channels, are inherently limited in spatial complexity and integration potential. In contrast, the additive approach demonstrated here enables freeform capillary architectures to be fabricated directly within microfluidic devices without reliance on sacrificial layers or extensive assembly. This capability opens opportunities for more advanced 3D cell culture models, as cells can migrate into and interact with the scaffold lattice, potentially guiding cellular organization and enhancing physiological relevance. The 3D scaffold surrounding the capillary membrane structure can also be engineered to have microscale or nanoscale surface topography to promote cell adhesion, differentiation, migration, and growth. Furthermore, by reducing the use of PDMS relative to conventional microfluidic chips, this system avoids small molecule leaching issues and creates a more biochemically compatible environment for sensitive biological assays.

A novel microfluidic interfacing strategy was also developed, utilizing heat shrink tubing and hypodermic needles to produce reliable and low-cost connections between the external fluidic system and the printed scaffold. This approach eliminated the need for bulky adapters and enabled a compact chip design. SEM analysis confirmed that the heat shrink tubing formed tight seals without intruding into the scaffold lattice regions, preserving perfusion pathways. Perfusion experiments, performed using a push-pull syringe pump and PIV analysis, validated the suitability of this system for future cell studies.

OrmoComp, PEGDA 700, and IP-PDMS all supported hNSC adhesion and viability, each exceeding 70% viability after three days in culture. Among the peripheral chip components, PLA lids demonstrated good compatibility, supporting healthy cell attachment and viability comparable to standard culture substrates. In contrast, LCD 3D-printed resins used for chip lids—despite not contacting the media directly—exhibited significant cytotoxicity, likely due to volatile compound release or insufficient post-curing. Although Siraya Tech Blu resin nominally meets ISO 10993-5 biocompatibility standards, the curing conditions applied in this study proved inadequate. These findings underscore that even non-contact components must be carefully evaluated to avoid adverse effects on sensitive neural cultures.

Conclusions: In this invention, we disclose a versatile and modular platform for fabricating perfusable microvascular scaffolds integrated within microfluidic chips using TPL. Leveraging mesh-based design, multi-material printing, and an accessible heat-shrink tubing interface, we successfully produced customizable 3D capillary networks with tunable mechanical properties.

Rigid (OrmoComp), moderately stiff (PEGDA 700), and soft elastomeric (IP-PDMS) materials were all successfully printed into a multi-material chip configuration. However, PEGDA 700 and IP-PDMS printed structures suffered from deformation after development, requiring a cone-based support structure to maintain fidelity. Flow validation demonstrated that the printed scaffolds supported perfusion at physiologically relevant flow speeds.

Cytocompatibility assays further confirmed that all TPL-printed scaffold materials supported robust human neural stem cell viability and attachment, validating their suitability for neurovascular and brain-on-chip applications. PLA lids demonstrated suitable biocompatibility, while LCD resin lids exhibited significant cytotoxicity, underscoring the importance of carefully selecting and processing all chip components to ensure compatibility with sensitive biological environments.

Together, these results establish a versatile and adaptable platform for creating biologically relevant 3D vascular scaffolds in microfluidic systems. Future efforts will focus on incorporating brain endothelial cells to create biomimetic BBB models and integrating microcapillary scaffolds into brain organoid systems to promote maturation and reduce necrosis. These advancements will expand the platform's utility for neurovascular modeling, disease research, and drug transport studies.

The scaffold structures described herein are fabricated using materials compatible with TPL or other high-resolution additive manufacturing methods. In specific embodiments, fabrication has been demonstrated using:
Poly(ethylene glycol) diacrylate (PEGDA): a hydrophilic, biocompatible resin offering stiffness suitable for structural fidelity while supporting neural stem cell attachment and differentiation;
IP-PDMS (photopolymerizable polydimethylsiloxane): a TPL-compatible elastomeric hybrid resin enabling fabrication of resilient, freestanding capillary-like structures; and
Ormocomp: a commercially available hybrid acrylate resin used for its low-shrinkage, structural rigidity, and compatibility with femtosecond laser TPL platforms.

These materials provide mechanical and optical properties suitable for microscale fabrication of perfusable vasculature, structured scaffolds, and microfluidic interfaces relevant to in-vitro brain models, particularly for supporting human neural stem cell culture and NVU co-culture. Additional photopolymerizable or TPL-compatible biomaterials may be employed, either alone or in hybrid form, depending on the targeted application. For brain modeling, softer or ECM-mimetic materials may better recapitulate tissue stiffness and biochemical cues, including: Gelatin methacryloyl (GelMA); Methacrylated hyaluronic acid (HAMA); Methacrylated collagen; Poly(ethylene glycol) dimethacrylate (PEGDMA); Poly(2-hydroxyethyl methacrylate) (pHEMA); and PEG-fibrinogen composites.

The microfluidic and scaffold platform is not limited to neural tissues. For modeling other tissues or organ systems, material choice may be tuned for biomechanical and cellular compatibility:
Cardiac tissue: GelMA, PDMS-GelMA hybrids, alginate-methacrylate
Liver: PEG-fibrinogen, chitosan-methacrylate, thiol-ene gels
Kidney/Gut epithelium: HAMA, collagen-MA, silk methacrylates

All such materials may be chosen to be biodegradable or non-biodegradable, and their crosslinking kinetics, swelling behavior, and stiffness may be customized via formulation and laser parameters. The customizable material and architectural framework makes this platform broadly adaptable for organ-on-chip, disease modeling, and regenerative engineering.

It should be understood that the full scope of the invention is not limited to the examples provided above. Additional features, advantages, and applications of the present invention are described below.

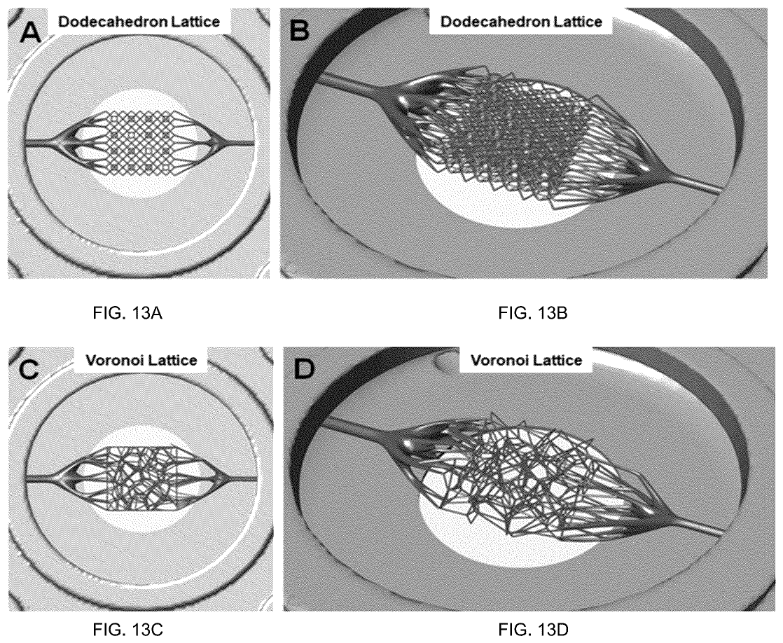

Tailorable and Path-Adaptable Design Platform: The scaffold's design is generated using Rhinoceros3D software, a visual programming tool that allows users to create custom geometries based on input parameters. The scaffold design is defined as a 3D path represented by a set of (x, y, z) coordinates, which determine the structure of the vascular membrane. The system can generate either a single-tube path (FIGS. 1C, 1E, and 1F) for continuous flow or a branched tube path (FIGS. 13A-13D) for more complex vascular networks.

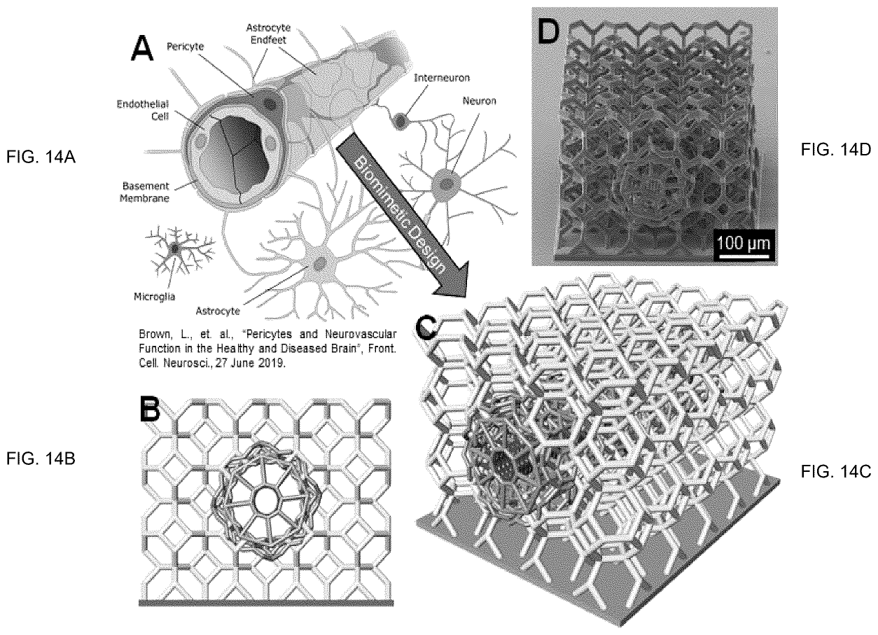

Coaxial Porous Membrane and Support Structures: The scaffold integrates a coaxial porous membrane surrounding the microvasculature (FIGS. 14A-14D), creating a compartmentalized space for pericytes, which are crucial for maintaining NVU integrity. The Crystallon add-in generates lightweight support beams that stabilize the capillary and coaxial membranes, allowing proper alignment of cellular structures while maintaining open channels for fluid flow. The support beams minimize internal space usage, ensuring that the capillaries remain functional without obstruction. The size and shape of the coaxial membrane can be customized according to experimental requirements, with examples including an inner diameter of 120 μm surrounding a 40 μm capillary, which reduces to ~20-25 μm when brain microvascular endothelial cells (BMECs) are seeded inside (FIG. 14D). The coaxial membrane facilitates interactions between BMECs, pericytes, astrocytes, and neurons, closely mimicking the natural biological processes of the NVU. Pericytes are compartmentalized within the coaxial membrane, while astrocytes are encouraged to send their end feet toward the capillaries, accurately recreating the BBB's function. When biodegradable materials are used, the scaffold gradually degrades, with the ECM secreted by cells taking over its structural role, ensuring the scaffold evolves into a fully biological tissue model for long-term studies.

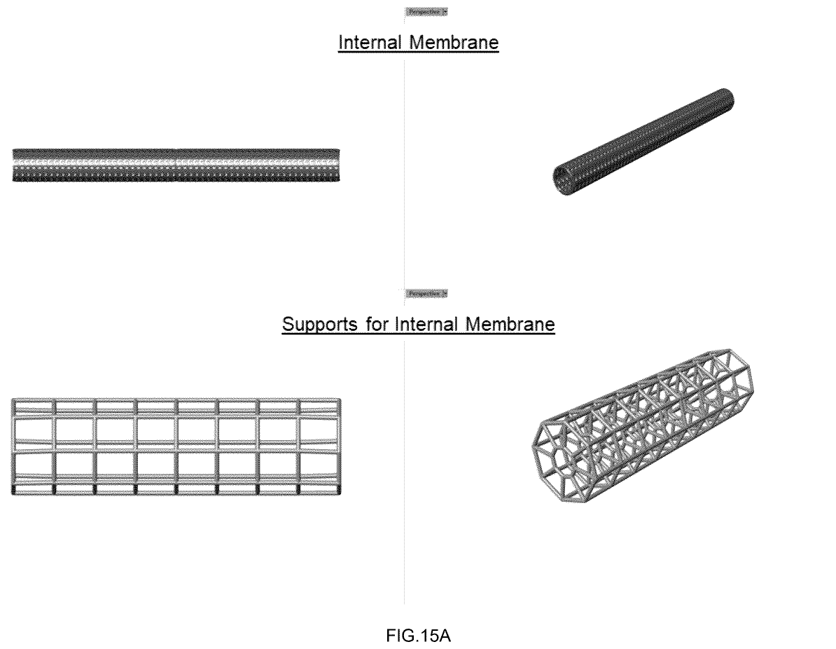

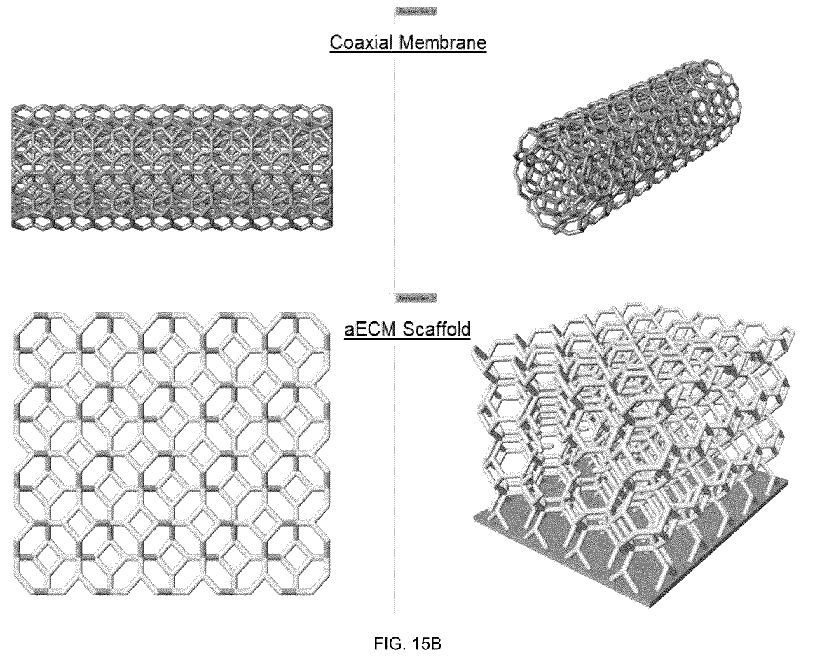

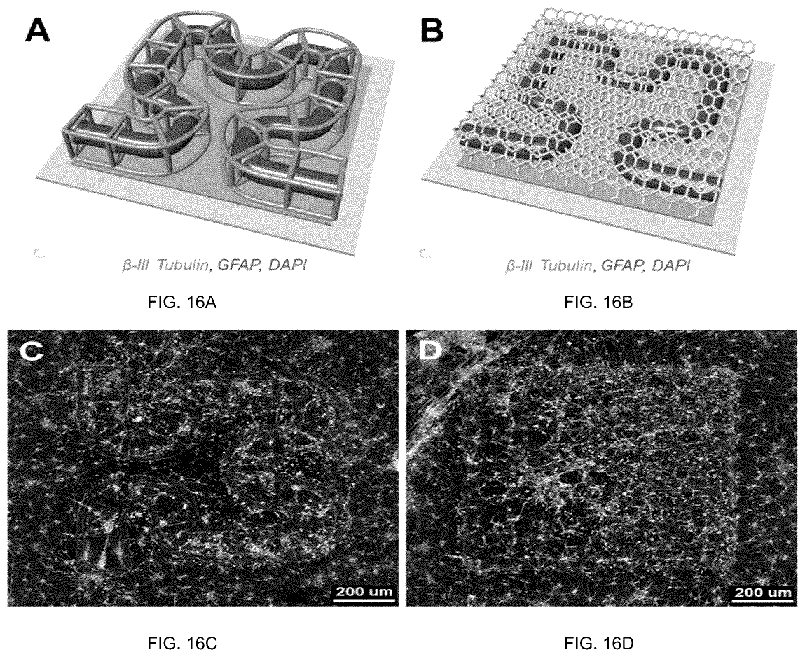

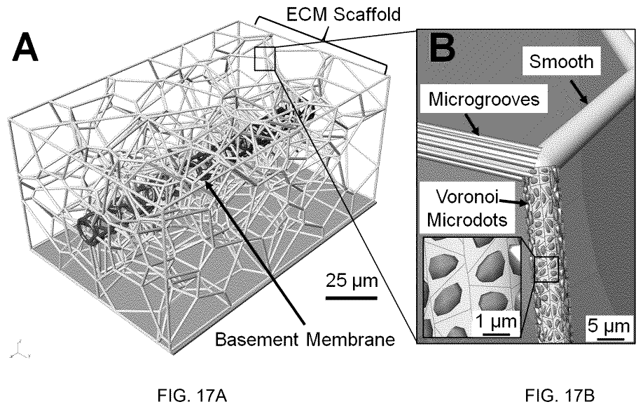

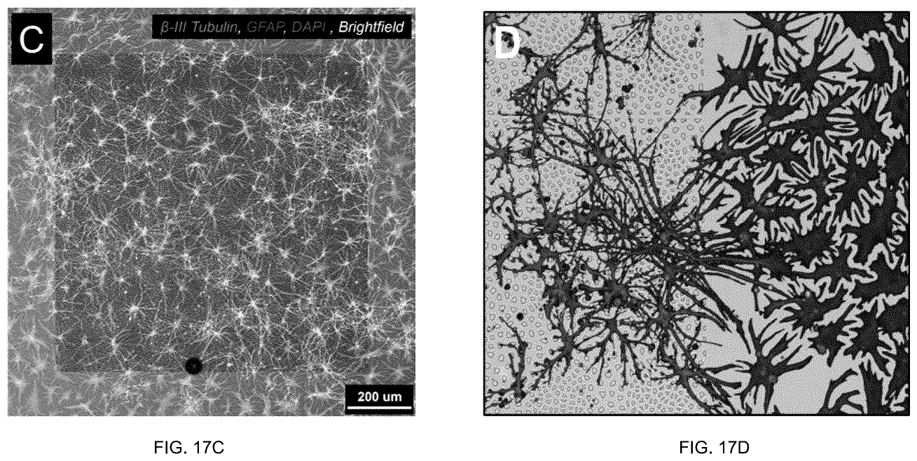

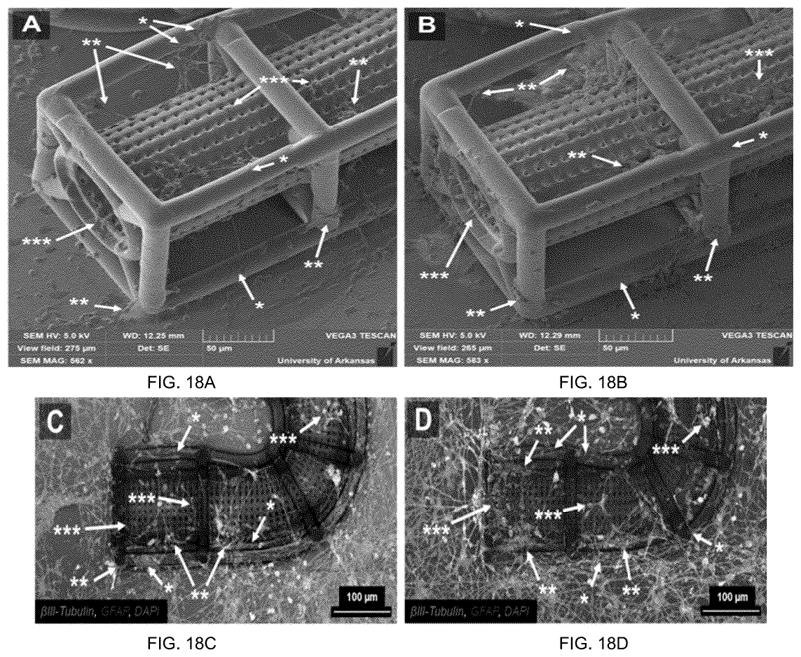

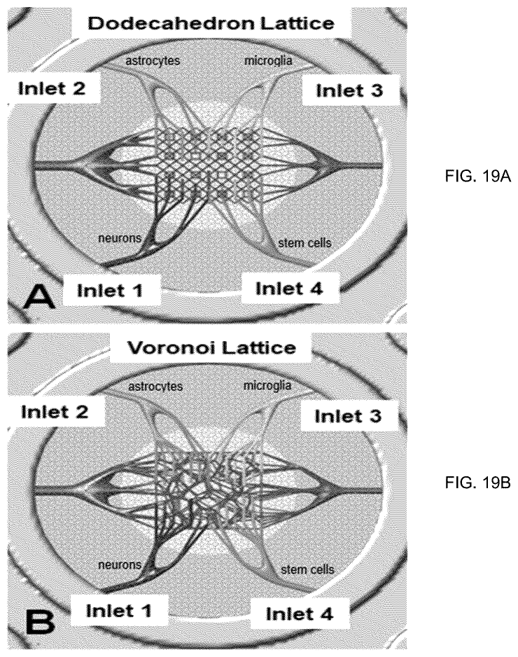

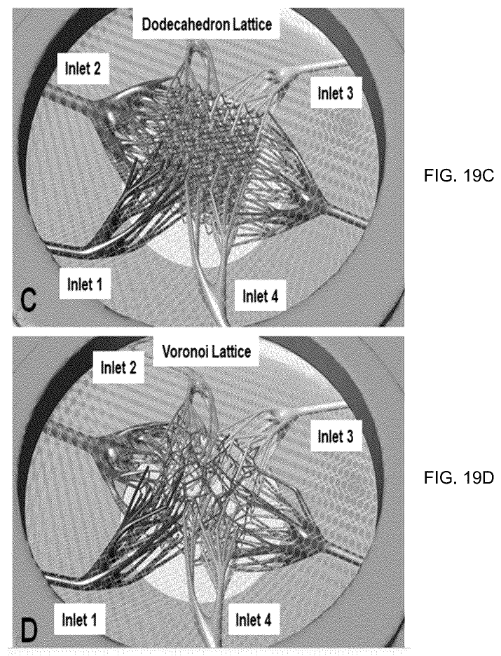

Customizable Support Lattice for Astrocytes and Neurons: The scaffold's support lattice is designed to promote differentiation of neural stem cells into neurons and astrocytes (FIG. 14B-14D). Details of individual components of FIG. 14C is illustrated in FIG. 15A-15B. Neurons differentiate on the scaffold's 3D surfaces, while astrocytes primarily form on the flat areas of the design (FIGS. 16C-16D), allowing the astrocyte-to-neuron ratio to be tailored for different brain regions. Furthermore, micro- and nanoscale topographical cues can be added to guide the organization, growth, and alignment of cells, ensuring the tissue model mimics in-vivo conditions (FIGS. 17A-17D). SEM images (FIGS. 18A-18D) show selective differentiation of hNSCs (human neural stem cells) on the vascularized lattice, with networks of neuronal extensions developing on the capillary structures. Astrocytes are primarily observed in flat areas, but some astrocytes were noted near the capillary, moving toward the scaffold. Two-photon lithography ensures high precision when fabricating the intricate topographical features necessary for guiding cell growth (FIG. 19A-D). This capability allows the scaffold to model different brain regions, offering flexibility for studying region-specific behaviors and disease conditions.

Compartmentalized Channels for Fluid Flow and Chemical Stimulation: The scaffold integrates compartmentalized microfluidic channels to provide precise control over the delivery of fluids, nutrients, cells, and chemical agents (e.g., alcohol, caffeine, drugs) (FIG. 19A-19D). These channels simulate the fluid dynamics of the brain's native microenvironment, ensuring continuous perfusion of the scaffold and maintaining cell viability.

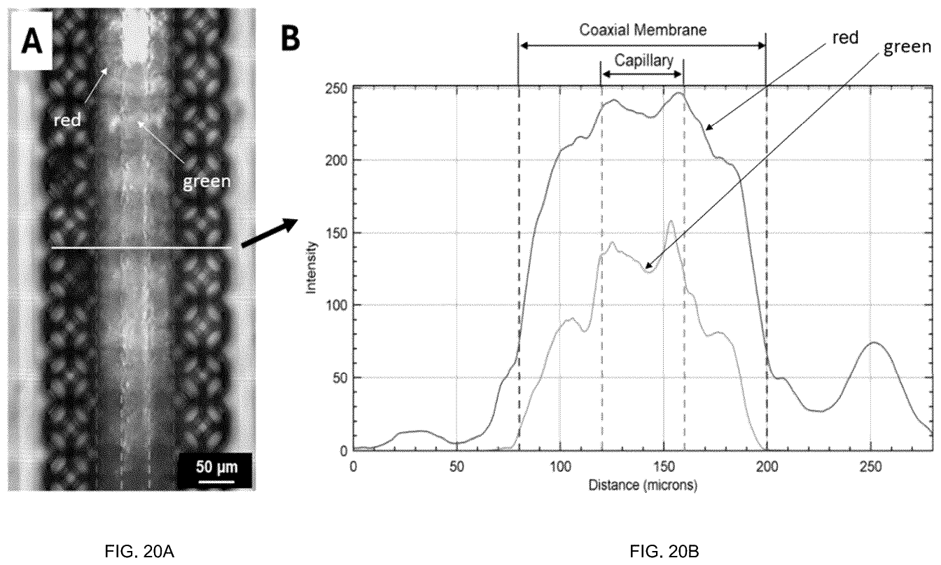

FIG. 20A-20B illustrates the flow of fluorescent tracers within the coaxial membrane chip, with green microparticles circulating through the interior capillary membrane and near-infrared (NIR) tracers flowing through the coaxial membrane. The fluorescence intensity profiles (red-green, RG) correspond to the tracer locations, confirming independent flow within each membrane channel and validating the functionality of the dual-channel system.

Key advantages of the present invention include its scalability, the customizable nature of the scaffold to fit specific experimental needs, organizational control of differentiating cell types, and its ability to support long-term perfusion. By integrating microfluidic channels and a robust fluid delivery system, the platform enables precise control over the delivery of nutrients, cells, and chemical agents, facilitating the study of drug responses and disease progression. This system is also capable of accommodating various cell types, such as brain microvascular endothelia cells (BMECs), astrocytes, neurons, microglia, and pericytes, making it a powerful tool for replicating the NVU in high-fidelity in-vitro conditions.

Additionally, the platform offers flexibility in material use, including biodegradable and non-biodegradable options, which makes it adaptable for long-term studies and dynamic tissue models that evolve as the scaffold degrades. The technology holds promise for a variety of applications, particularly in the fields of pharmaceutical development, personalized medicine, and neurodegenerative disease research, making it a valuable tool for academic and industrial research environments.

Applications of the present invention include:
Neurovascular Drug Testing Platforms: This invention could be used to create advanced in-vitro drug testing platforms for pharmaceutical companies. These platforms would allow for more accurate testing of therapies targeting the blood-brain barrier (BBB), neurodegenerative diseases, and brain cancers.
Customizable Organ-on-a-Chip Systems: A product offering customizable organ-on-a-chip solutions, where the system is tailored to different brain regions or disease states, could benefit companies working in personalized medicine or advanced clinical research.
Tissue Engineering Scaffolds: The 3D vascularized scaffolds could be adapted for tissue regeneration and repair products, supporting companies in the regenerative medicine industry or those developing implants that need vascularization.
Neuroscience Research Kits: Academic institutions and research laboratories could use this platform as a kit for conducting neuroscience experiments, allowing them to simulate various brain conditions or study the effects of different drugs and chemical agents on brain tissue.
Personalized Medicine Platforms: The ability to customize the scaffold design for specific patient models could lead to products that help screen treatments for patients with specific neurological diseases, optimizing drug effectiveness for personalized care.
High-Fidelity Cell Culture Systems: A more general application could be as cell culture systems with long-term perfusion capabilities, offering a more physiologically relevant environment for general biological research, stem cell studies, and preclinical testing.
Microfluidic Diagnostic Devices: This platform could be adapted into diagnostic devices for medical laboratories, enabling more accurate detection of brain-related diseases by replicating patient-specific brain conditions in a controlled environment.
Neurotoxicity Screening Services: A service that uses this platform to offer neurotoxicity screening for new chemical agents or environmental toxins could be valuable for environmental health agencies and regulatory bodies looking to assess risks to human health.
Bioreactor Components for Long-Term Cultures: The heat-shrink tubing interface and microfluidic channels make this platform ideal as a bioreactor component for companies developing long-term culture systems for diverse tissue types, not just brain tissue.
Stem Cell Differentiation Products: The scaffold could be used to develop products for stem cell differentiation, helping companies or research groups working in cell lineage studies or cell-based therapies.
Advanced Cosmetic Testing Platforms: Outside of the pharmaceutical field, this platform could potentially be used for cosmetic testing, providing a more ethical and accurate alternative to animal testing, particularly in testing how substances affect skin-brain barrier interactions.

Additional advantages of the present invention include:
1. Realistic Simulation of Human Brain Tissue: Unlike traditional in-vitro models, this platform offers a three-dimensional scaffold that closely replicates the intricate structure and function of the neurovascular unit (NVU), including the blood-brain barrier (BBB). This level of physiological accuracy provides superior data for drug testing and disease modeling.
2. Customizability and Scalability: The scaffold can be tailored to fit specific experimental needs. Researchers can adjust vascular diameter, pore size, and material properties, making it versatile for various research applications. The system supports both single-tube and branched vascular designs, allowing for greater flexibility in experimental setup.
3. Long-Term Perfusion Capability: The platform integrates a heat-shrink tubing interface that allows it to connect seamlessly with external pumps for controlled, long-term perfusion of fluids, nutrients, and chemicals. This ensures the sustainability of cell cultures and the ability to conduct prolonged experiments.
4. Multi-Cell Co-Culture Support: The scaffold is designed to organize and support the co-culture of multiple cell types found in the NVU, including BMECs, astrocytes, neurons, microglia, and pericytes. This ability to recreate the complex interactions within the brain's microenvironment enhances the platform's utility for high-fidelity neurovascular research.
5. Material Flexibility: The system accommodates both biodegradable and non-biodegradable materials, allowing for dynamic tissue modeling. As biodegradable materials degrade, they can be replaced by the extracellular matrix (ECM) secreted by cells, making the system suitable for long-term studies where tissue evolves naturally over time.
6. Improved Drug Testing Accuracy: By mimicking the BBB and enabling compartmentalized delivery of drugs and nutrients, this platform provides a more accurate environment to test drug efficacy and toxicity. This feature helps reduce false positives/negatives often seen in simpler models, increasing the translatability of preclinical research to clinical settings.
7. Advanced Microfluidic Channels: The integration of compartmentalized microfluidic channels allows precise delivery of fluids, nutrients, and chemical agents. This feature supports complex experiments, such as testing the impact of drugs on different parts of the scaffold or simulating disease conditions.
8. Innovative Fabrication Technology: The scaffold is fabricated using TPL, which offers nanoscale resolution and the ability to create real-scale capillaries. This advanced technique surpasses conventional methods like photolithography, enabling the creation of highly detailed and functional vascular structures.
9. Broad Application Scope: The platform is applicable to a wide range of research areas, including neurodegenerative diseases, drug discovery, personalized medicine, and pharmaceutical development. Its precision and flexibility make it ideal for both academic research and industrial applications.
10. Potential for Commercialization: With its innovative design, material versatility, and long-term experimental capabilities, the platform holds significant commercial potential, particularly for companies focused on biotech, pharmaceuticals, and tissue engineering.

The present invention has been described with reference to certain preferred and alternative embodiments that are intended to be exemplary only and not limiting to the full scope of the present invention as set forth in the appended claims.

We claim:

1. A microfluidic chip for use in in-vitro brain models, comprising:
a microcapillary scaffold comprises a scaffold base and a scaffold vasculature, wherein said scaffold base comprises a first port and a second port;
a microfluidic interface system attached to said microcapillary scaffold, wherein said microfluidic interface system comprises a first heat-shrink tubing, a first hypodermic needle, a second heat-shrinking tubing and a second hypodermic needle, wherein a first end of said first heat-shrink tubing is attached to said first port of said scaffold base and a first end of said second heat-shrink tubing to said second port of said scaffold base, wherein said first hypodermic needle is attached to a second end of said first heat-shrink tubing and said second hypodermic needle is attached to a second end of said second heat-shrink tubing;
an external fluidic system connected to said microfluidic interface system for providing fluid for perfusion through said microcapillary scaffold; and
a plate comprising a well, wherein said microcapillary scaffold is secured in said well.

2. The microfluidic chip of claim 1, wherein said microcapillary scaffold comprises a capillary membrane and a polygonal support.

3. The microfluidic chip of claim 1, wherein said microcapillary scaffold comprises a capillary membrane and a lattice support.

4. The microfluidic chip of claim 2, wherein said capillary membrane is curved or branched.

5. The microfluidic chip of claim 1, wherein said scaffold base is made from a rigid hybrid polymer resin and said scaffold vasculature is made from said rigid hybrid polymer resin, a PEGDA resin, an elastomeric resin, or an extracellular matrix-mimetic material.

6. The microfluidic chip of claim 1, wherein a retaining wall separates said scaffold vasculature and said first port.

7. The microfluidic chip of claim 5, wherein said scaffold base comprises a plurality of support cones.

8. The microfluidic chip of claim 7, wherein said plurality of support cones support said scaffold vasculature made from said PEGDA resin or said elastomeric resin or said extracellular matrix-mimetic material.

9. The microfluidic chip of claim 1, wherein said plate comprises a first holder for said first hypodermic needle and a second holder for said second hypodermic needle.

10. The microfluidic chip of claim 1, wherein said external fluidic system comprises a pump.

11. The microfluidic chip of claim 2, wherein a coaxial membrane surrounds said capillary membrane.

12. A method of making a microfluidic chip for use in in-vitro brain models, said method comprising the steps of:
fabricating a microcapillary scaffold using two photon lithography, wherein said microcapillary scaffold comprises a scaffold base and a scaffold vasculature, wherein said scaffold base comprises a first port and a second port;
attaching a microfluidic interface system to said microcapillary scaffold, wherein said microfluidic interface system comprises a first heat-shrink tubing, a first hypodermic needle, a second heat-shrinking tubing and a second hypodermic needle, wherein said step of attaching said microfluidic interface system comprises the step of attaching a first end of said first heat-shrink tubing to said first port of said scaffold base and applying heat sufficient to seal said first end of said first heat-shrink tubing to said first port, wherein said step of attaching said microfluidic interface system further comprises the step of attaching a first end of said second heat-shrink tubing to said second port of said scaffold base and applying heat sufficient to seal said second end of said second heat-shrink tubing to said second port, wherein said step of attaching said microfluidic interface system further comprises the step of inserting said first hypodermic needle in a second end of said first heat-shrink tubing and inserting said second hypodermic needle in a second end of said second heat-shrink tubing; and
securing said microcapillary scaffold in a well of a plate.

13. The method of claim 12, further comprising the step of creating a design of said microcapillary scaffold before said step of fabricating said microcapillary scaffold.

14. The method of claim 12, wherein said step of creating a design of said microcapillary scaffold comprises the step of using three-dimensional modeling software.

15. The method of claim 12, wherein said microcapillary scaffold comprises a capillary membrane and a polygonal support.

16. The method of claim 12, wherein said microcapillary scaffold comprises a capillary membrane and a lattice support.

17. The method of claim 12, wherein said step of fabricating said microcapillary scaffold using two step lithography comprises the step of using a rigid hybrid polymer resin to fabricate at least some of said microcapillary scaffold.

18. The method of claim 17, wherein said step of fabricating said microcapillary scaffold using two step lithography comprises the step of using PEGDA resin or an elastomeric resin to fabricate some of said microcapillary scaffold.

19. The method of claim 18, wherein said rigid hybrid polymer resin is used to fabricate said scaffold base of said microcapillary scaffold and wherein said PEGDA resin or said elastomeric resin is used to fabricate said scaffold vasculature of said microcapillary scaffold.

20. The method of claim 12, further comprising the step of covering said well with a glass lid.
